SEQUENCE LISTING (2 sequences)

SEQ ID NO: 1 - DNA, 276 bp, Homo sapiens
  source 1..276: mol_type = genomic DNA
atggctcctactagctcgagcactaagaaaactcaactgcaattggagcatctgctgctggatctgcagatgattctgaatggcatcaataactacaagaaccctaagctgactcgcatgctgactttcaaattctacatgccgaaaaaggctaccgagctcaaacatctccagtgcctggaagaggaactgaagccgctggaggaagtacttaacctggcacagtctaagaacttccacctgcgtccgcgtgacctgatctccaacatcaagtaa

SEQ ID NO: 2 - protein, 273 aa, Homo sapiens
METALAPROTHRSERSERSERTHRLYSLYSTHRGLNLEUGLNLEUGLUHISLEULEULEUASPLEUGLNMETILELEUASNGLYILEASNASNTYRLYSASNPROLYSLEUTHRARGMETLEUTHRPHELYSPHETYRMETPROLYSLYSALATHRGLULEULYSHISLEUGLNCYSLEUGLUGLUGLULEULYSPROLEUGLUGLUVALLEUASNLEUALAGLNSERLYSASNPHEHISLEUARGPROARGASPLEUILESERASNILELYS

(19) United States
(12) Patent Application Publication
(10) Pub. No.: US 2026/0183346 A1
(43) Pub. Date: Jul. 2, 2026

(54) LIVER DISEASE TREATMENTS USING ATTENUATED STRAINS OF SALMONELLA TYPHIMURIUM

(71) Applicant: Salspera, LLC, Oakdale, MN (US)

(72) Inventors: Aydin Moradian, Sunfish Lake, MN (US); Daniel Saltzman, Mendota Heights, MN (US)

(21) Appl. No.: 19/289,882

(22) Filed: Aug. 4, 2025

Related U.S. Application Data
(60) Provisional application No. 63/678,946, filed on Aug. 2, 2024.

(51) Int. Cl. A61K 35/74; A61K 38/00; A61K 45/06; A61P 35/00; C07K 14/55; C12N 15/74

(52) U.S. Cl. CPC A61K 35/74; A61K 45/06; A61P 35/00; C07K 14/55; C12N 15/74; A61K 38/00

(57) ABSTRACT

The composition and methods of treatment disclosed or contemplated herein include compositions and methods of treatment for liver-related diseases. The overall treatment strategy would involve the identification of a target protein or receptor within the liver that is responsible for ongoing liver injury. The strain of orally administered Salmonella Typhimurium that colonizes the liver would be engineered to secrete a particular protein or nanobody that would stop and/or reverse the progressive hepatic disease

### CROSS-REFERENCE TO RELATED APPLICATION(S)

This application claims the benefit under 35 U.S.C. § 119 (e) to U.S. Provisional Application 63/678,946, filed Aug. 2, 2024 and entitled “LIVER DISEASE TREATMENTS USING ATTENUATED STRAINS OF SALMONELLA TYPHIMURIUM,” which is hereby incorporated herein by reference in its entirety.

### SEQUENCE LISTING

The instant application contains a Sequence Listing that has been submitted in ASCII format via EFS-Web and is hereby incorporated by reference in its entirety. The ASCII copy, created on Nov. 10, 2025, is named 849776WOU1_SequenceListing 2025.11.10.xml, and is 3,300 bytes in size.

### FIELD

The various embodiments herein relate to the treatment of liver diseases using attenuated strains of Salmonella Typhimurium containing one or more plasmids carrying a coding sequence expressing proteins, nanobodies or pathway inhibitors.

### BACKGROUND

Isolated liver diseases, which include nonalcoholic steatohepatitis (NASH) or alcoholic hepatic cirrhosis, remains extremely difficult to treat for a variety of reasons (See Tapper, E B, Parikh N D. Diagnosis and Management of Cirrhosis and Its Complications: A Review. JAMA.2023; 329(18):1589-1602. doi:10.1001/jama.2023.5997; Kockerling D, Nathwani R, Forlano R, Manousou P, Mullish B H, Dhar A. Current and future pharmacological therapies for managing cirrhosis and its complications. World J Gastroenterol. 2019 Feb. 28; 25(8):888-908. doi: 10.3748/wjg.v25.i8.888. PMID: 30833797; PMCID: PMC6397723; and Tapper E B, Ufere N, Huang Q, Loomba R. Review article: Current and emerging therapies for the management of cirrhosis and its complications. Aliment Pharmacol Ther. 2022; 55:1099-1115). All therapies for these hepatic diseases focus on the secondary complications of hepatic fibrosis. For example, diuretics to treat ascites related to portal hypertension from hepatic fibrosis and cirrhosis or beta blockers, sclerotherapy, and intestinal vasoactive medications to treat variceal disease that is the result of hepatic fibrosis and cirrhosis. There have been a number of clinical trials for agents that attempt to address the hepatic fibrosis itself with the aim to reverse or prevent this organ specific fibrosis, however, there has been no meaningful success in this field. The primary reason for the lack of any therapies for these diseases is the lack of a mechanism to deliver a therapy directly to the liver and avoid systemic exposure. Up to now, the only means to deliver a particular therapy directly to the liver are direct portal vein infusion and/or hepatic arterial infusion pumps. These therapies have been proven to be extremely impractical, pose a significant risk to patients given how morbid portal vein or hepatic arterial cannulation #3277290 can be, and not easy to employ. Thus, there is a desperate need for an agent that can treat hepatic specific diseases that avoid systemic exposure and potential toxicities.

It has been well described that attenuated strains of Salmonella Typhimurium, such as Saltikva® (Salspera) strains, colonize the liver in a robust pattern where these bacteria have been seen within the hepatic Kupffer cells, hepatocytes, interstitial spaces between the various hepatic cellular populations, and within hepatic tumors themselves. Saltikva® is an orally administered attenuated strain of Salmonella Typhimurium that carries the human gene for interleukin-2 (IL-2). There are extensive preclinical and clinical studies with these attenuated strains that demonstrate clear colonization of the liver and the delivery of IL-2 to the liver and metastatic tumor microenvironment. For example, Saltikva strains has been shown to safely colonize the liver for up to 6 weeks after a single oral dose of 10⁹ bacteria in a murine model. Histologically speaking, these bacteria were observed to colonize the hepatocytes themselves, hepatic macrophages, and the interstitial cellular spaces without any toxicity (see Sorenson B S, Banton K L, Augustin L B, Frykman N L, McCulloch K A, Bulander R D, Wasiluk K, Leonard A S, Saltzman D A. Safety of an Attenuated Strain of Salmonella Typhimurium Expressing C-terminal Truncated Human IL-2: A Murine Infection Model. Biologics: Targets and Therapy. 2010 4:61-73).

There is a need in the art for a delivery system with an expanded range of treatments capable of expressing additional proteins, inhibitors, receptors or nanobodies that would be beneficial for the treatment of additional liver diseases.

### BRIEF SUMMARY

Discussed herein are various compositions and methods of treatment for liver-related diseases. The overall treatment strategy would involve the identification of a target protein or receptor within the liver that is responsible for ongoing liver injury. The strain of orally administered Salmonella Typhimurium that colonizes the liver would be engineered to secrete a particular protein or nanobody that would stop and/or reverse the progressive hepatic disease.

While multiple embodiments are disclosed, still other embodiments will become apparent to those skilled in the art from the following detailed description, which shows and describes various illustrative implementations. As will be realized, the various embodiments herein are capable of modifications in various obvious aspects, all without departing from the spirit and scope thereof. Accordingly, the drawings and detailed description are to be regarded as illustrative in nature and not restrictive.

Example 1 comprises a liver treatment agent comprising an effective amount of attenuated Salmonella typhimurium containing a plasmid carrying a coding sequence encoding one or a combination of inhibitors, antagonists or nanobodies capable of reducing fibroid expression

Example 2 comprises a liver treatment agent comprising an effective amount of attenuated Salmonella typhimurium containing a plasmid carrying a coding sequence encoding a therapeutic composition, said coding sequence directed to one or more of a TGF-β inhibitor, a PDGF inhibitor, a CTGF inhibitor, a TIMP inhibitor, a T-1 receptor antagonist, an NF-κB inhibitor or a periostin inhibitor.

Example 3 comprises the liver treatment agent of Example 2 wherein the coding sequence is directed to a PDGF inhibitor.

Example 4 comprises the liver treatment agent of Example 2 wherein the coding sequence is directed to a CTGF inhibitor.

Example 5 comprises the treatment agent of Example 2 wherein the coding sequence is directed to a TIMP inhibitor.

Example 6 comprises the liver treatment agent of Example 2 wherein the coding sequence is directed to an ET-1 receptor antagonist

Example 7 comprises the liver treatment agent of Example 2 wherein the coding sequence is directed to a NF-κB inhibitor.

Example 8 comprises the liver treatment agent of Example 2 wherein the coding sequence is directed to a periostin inhibitor.

Example 9 comprises the liver treatment agent of Example 2 wherein the coding sequence is directed to a TGF-β inhibitor.

Example 10 comprises the liver treatment agent of Example 2 wherein the agent is capable of reducing fibroid expression.

Example 11 comprises the liver treatment agent of Example 2 wherein the agent is taken as an oral dose.

Example 12 is the liver treatment of Example 11 wherein the oral dose is 10⁹ bacteria

Example 13 is a liver treatment agent comprising an effective amount of one or more attenuated Salmonella strains wherein at least one strain contains a plasmid carrying a coding sequence encoding one or a combination of inhibitors, antagonists or nanobodies capable of reducing fibroid expression.

Example 14 is the liver treatment agent of Example 13 wherein the Salmonella strains are Salmonella typhimurium strains.

Example 15 is the liver treatment agent of Example 14 wherein the agent comprises at least two strains contain a plasmid carrying a coding sequence encoding one or a combination of inhibitors, antagonists or nanobodies capable of reducing fibroid expression, wherein the coding sequence for the at least two strains encode for a different inhibitor, antagonist or nanobody, or a different combination of inhibitors, antagonists or nanobodies.

Example 16 is the liver treatment agent of Example 15 wherein the inhibitors, antagonists or nanobodies are selected from the group comprising a TGF-β inhibitor, a PDGF inhibitor, a CTGF inhibitor, a TIMP inhibitor, a T-1 receptor antagonist, an NF-κB inhibitor or a periostin inhibitor.

Example 17 is the liver agent of Example 1 wherein the attenuated Salmonella typhimurium containing a plasmid carrying a coding sequence encodes a protein inhibitor

Example 18 is the liver agent of Example 1 wherein the attenuated Salmonella typhimurium containing a plasmid carrying a coding sequence encodes a receptor inhibitor

Example 19 is the liver agent of Example 1 wherein the agent is capable of reducing fibroid expression wherein the fibroid expression is a complication from nonalcoholic steatohepatitis (NASH).

Example 20 is the liver agent of Example 1 wherein the agent is capable of reducing fibroid expression wherein the fibroid expression is a complication from alcoholic hepatic cirrhosis

### BRIEF DESCRIPTION OF THE DRAWINGS

FIG. 1 shows the pIL2 plasmid containing the coding sequence encoding the human interleukin-2 protein inserted into Salmonella typhimurium χ4550pIL2.

FIG. 2 shows the amino acid sequence of a DNA encoding normal human interleukin-2.

FIG. 3 shows the DNA sequence of the normal human interleukin-2 protein.

FIG. 4 is a bar graph representing (Tumor Treatment Model) reduced hepatic metastases in response to control (saline), Salmonella typhimurium χ4550 and Salmonella typhimurium χ4550pIL2 with the truncated human IL-2 gene.

FIG. 5 shows a bar graph representing (Tumor Treatment Model) reduced hepatic tumor number in response to control (saline), Salmonella typhimurium χ4550 and Salmonella typhimurium χ4550pIL2 with the truncated human IL-2 gene.

FIG. 6 shows a bar graph representing (Tumor Treatment Model) reduced hepatic tumor volume in response to control (saline), Salmonella typhimurium χ4550 and Salmonella typhimurium χ4550pIL2 with the truncated human IL-2 gene.

FIG. 7 shows a bar graph representing (Tumor Treatment Model) elevated hepatic NK cells in response to control (saline), Salmonella typhimurium χ4550 and Salmonella typhimurium χ4550pIL2 with the truncated human IL-2 gene.

FIG. 8 shows a bar graph representing (Tumor Treatment Model) elevated hepatic CD8+ cells in response to control (saline), Salmonella typhimurium χ4550 and Salmonella typhimurium χ4550pIL2 with the truncated human IL-2 gene.

FIG. 9 shows the results of the four groups (control, oil, S. typhimurium χ4550pIL2 and S. typhimurium χ4550pIL2 plus antioxidant oil on total tumor number in tumor burdened mice. As shown, the S. typhimurium χ4550pIL2 plus antioxidant oil group had approximately an eight fold reduction in total number of tumor cells compared to the group receiving only oil. An approximate two fold reduction in the number of tumors was shown compared to the group receiving only S. typhimurium χ4550pIL2. Compared to the control group, the group receiving S. typhimurium χ4550pIL2 plus antioxidant oil showed a four fold reduction in the number of tumors.

FIG. 10 shows the results of the four groups (control, antioxidant oil, S. typhimurium χ4550pIL2 and S. typhimurium χ4550pIL2+ antioxidant oil) on tumor volume in tumor burdened mice. As shown the S. typhimurium χ4550pIL2 plus antioxidant oil group showed almost negligible tumor volume compared to the antioxidant oil only group, the control group, and the S. typhimurium χ4550pIL2 only group.

FIG. 11 shows the cumulative survival of tumor naive mice after being fed control, S. typhimurium χ4550 and S. typhimurium χ4550pIL2. The groups receiving S. typhimurium χ4550 and S. typhimurium χ4550pIL2 show an almost 40% long term survival rate increase over the control group.

FIG. 12 shows the results of the four groups (control, antioxidant oil, S. typhimurium χ4550pIL2 and S. typhimurium χ4550pIL2 plus antioxidant oil) on NK cell population in tumor naive mice. A statistically significant increase over the control group and antioxidant oil only group is shown in the S. typhimurium χ4550pIL2 and S. typhimurium χ4550pIL2 plus antioxidant oil groups.

FIG. 13 shows the results of the four groups (control, antioxidant oil, S. typhimurium χ4550pIL2 and S. typhimurium χ4550pIL2 plus antioxidant oil) on CD8+ cell population in tumor naive mice. A slight increase in CD8+ T cell population is shown in the mice in the S. typhimurium χ4550pIL2 plus antioxidant oil group.

FIG. 14 shows the results of the four groups (control, antioxidant oil, S. typhimurium χ4550pIL2 and S. typhimurium χ4550pIL2 plus antioxidant oil) on CD4+ T helper cell population in tumor naive mice. An overall statistically significant increase in CD4+ T helper cell population is shown in the S. typhimurium χ4550pIL2 plus antioxidant oil group.

FIG. 15 shows the results of the four groups (control, antioxidant oil, S. typhimurium χ4550pIL2 and S. typhimurium χ4550pIL2 plus antioxidant oil) on tumor number in tumor naive mice. A statistically significant decrease in tumor number is shown in the S. typhimurium χ4550pIL2 and S. typhimurium χ4550pIL2 plus antioxidant oil groups.

FIG. 16 shows the results of the four groups (control, antioxidant oil, S. typhimurium χ4550pIL2 and S. typhimurium χ4550pIL2 plus antioxidant oil) on tumor volume in tumor naive mice. A statistically significant decrease in tumor volume is observed in the S. typhimurium χ4550pIL2 group, with a further decrease observed in the S. typhimurium χ4550pIL2 antioxidant oil group.

FIG. 17 shows is a bar graph showing the reduction in tumor size of retroperitoneal neuromblastoma in control (saline), Salmonella typhimurium χ4550 and Salmonella typhimurium χ4550pIL2 with the truncated human IL-2 gene.

FIG. 18 is a bar graph showing the reduction in tumor volume of retroperitoneal neuromblastoma in control (saline), Salmonella typhimurium χ4550 and Salmonella typhimurium χ4550pIL2 with the truncated human IL-2 gene.

FIG. 19 is a bar graph showing the reduction in tumor weight of retroperitoneal neuromblastoma in control (saline), Salmonella typhimurium χ4550 and Salmonella typhimurium χ4550pIL2 with the truncated human IL-2 gene.

FIG. 20 is a bar graph showing the reduction in tumor number of pulmonary metastases from osteosarcoma in control (saline), Salmonella typhimurium χ4550 and Salmonella typhimurium χ4550pIL2 with the truncated human IL-2 gene.

FIG. 21 is a bar graph showing the reduction in number of hepatic metastases in mice orally administered Salmonella-IL2 vs. saline (control) or SaS Salmonella-no-IL2.

FIG. 22 is a bar graph showing the reduction of liver metastases in mice treated with Salmonella-IL2 where NK and CD8+ cell populations were depleted.

FIG. 23 is a bar graph showing that the invasion efficiency of Salmonella-IL2 is greatest in hepatocytes, but Salmonella-IL2 also invades various tumor cell lines.

FIG. 24 is a bar graph showing that the division efficiency of Salmonella-IL2 is greatest within neuroblastoma and osteosarcoma tumor cell lines over a 24-hour period compared with hepatoma and colon cancer cell lines.

FIG. 25 is a bar graph showing that the tumor volume (Panel A) and weight (Panel B) of retroperitoneal neuroblastoma in animals are reduced in mice treated with Salmonella-no-IL2 and Salmonella-IL2.

FIG. 26 is a bar graph showing serum cytokines at day 14 after gavage treatment in retroperitoneal neuroblastoma.

FIG. 27 is a bar graph representing the tumor volume of retroperitoneal neuroblastoma in animals being reduced in mice treated with Salmonella-no-IL2 and Salmonella-IL2.

FIG. 28 is a bar graph representing the tumor weight of retroperitoneal neuroblastoma in animals being reduced in mice treated with Salmonella-no-IL2 and Salmonella-IL2.

FIG. 29 is a bar graph representing serum cytokines at day 14 after gavage treatment in retroperitoneal neuroblastoma.

### DETAILED DESCRIPTION

The various composition and methods of treatment embodiments disclosed or contemplated herein include compositions and methods of treatment for liver-related diseases. The overall treatment strategy involves the identification of a target protein or receptor within the liver that is responsible for ongoing liver injury. The strain of orally administered Salmonella Typhimurium that colonizes the liver is then engineered to secrete a particular protein or nanobody that would stop and/or reverse the progressive hepatic disease.

It is well known that fibrosis in the liver or liver cirrhosis involves the excessive accumulation of extracellular matrix proteins, primarily collagen, leading to scar tissue formation. Progressive fibrosis in the liver leads to a multitude of health complications such as varices and variceal bleeds, hepatic encephalopathy, and ascites. Ultimately, the only known cure for hepatic cirrhosis is liver transplantation. The availability of organs to transplant, especially the liver and the costs of such a treatment, make this option of treatment not readily available or practical on a large scale. Targeting proteins involved in fibrogenesis can potentially prevent or reverse liver fibrosis and the devastating complications associated with liver fibrosis. Key proteins and pathways can be targeted for treatments.

Targeting proteins and pathways through methods and compositions of the various embodiments herein provides therapeutic strategies to mitigate liver fibrosis. An engineered attenuated strain of orally administered Salmonella Typhimurium according to any implementation herein can colonize the liver and subsequently secrete one or a combination of nanobodies that can block any of the proteins and/or their receptors described herein to stop and reverse the ongoing hepatic fibrosis. In addition, combination therapies targeting multiple pathways can provide synergistic effects and enhance efficacy in treating advanced liver fibrosis.

It is well described that attenuated strains of Salmonella Typhimurium, such as Saltikva® (Salspera) strains colonize the liver in a robust pattern where these bacteria have been seen within the hepatic Kupffer cells, hepatocytes, interstitial spaces between the various hepatic cellular populations, and within hepatic tumors themselves. Saltikva® is an orally administered attenuated strain of Salmonella Typhimurium that carries the human gene for interleukin-2 (IL-2). There are extensive preclinical and clinical studies with these attenuated strains that demonstrate clear colonization of the liver and the delivery of IL-2 to the liver and metastatic tumor microenvironment. For example, Saltikva strains has been shown to safely colonize the liver for up to 6 weeks after a single oral dose of 109 bacteria in a murine model. For reference, see:

Saltzman D A, Katsanis E, Hasz D E, Vigdorovich V, Curtiss III R E, Kelly S M, Anderson P M, and Leonard A S: Liver treatment Mechanisms of Attenuated Salmonella typhimurium Containing the Gene for Human Interleukin-2. Journal of Pediatric Surgery, 32(2):301-306, 1997.

Saltzman D A, Katsanis E, Heise C P, Hasz D E, Kelly S M, Curtiss R, Anderson P M, and Leonard A S: Hepatic and Splenic Colonization for the Attenuated Salmonella typhimurium Containing the Gene for Human Interleukin-2: A Novel Liver treatment Agent. Cancer Biotherapy and Radiopharmaceuticals, 12910:37-45, 1997.

Saltzman D A, Heise C P, Hasz D E, Zebede M, Kelly S M, Curtiss III R, Leonard A S, and Anderson P M. Attenuated Salmonella typhimurium Containing Interleukin-2 Decreases Number of MC-38 Hepatic Metastases: A Novel Liver treatment Agent. Cancer Biotherapy and Radiopharmaceuticals, 11(2)145-153, 1996

Soto L J, Sorenson B S, Kim A S, Feltis B A, Leonard A S, and Saltzman D A: Attenuated Salmonella typhimurium prevents the Establishment of Hepatic Colorectal Metastases and Improves Survival in a Murine Model. Tumor Biology 23:S1:30, 2002.

Feltis B A, Miller J S, Sahar D A, Kim A S, Saltzman D A, Leonard A S, Wells, C L, and Sielaff T D: Liver and Circulating NK1.1 (+) CD3 (−) Cells are Increased in Infection with Attenuated Salmonella typhimurium and are Associated with Reduced Tumor in Murine Liver Cancer. Journal of Surgical Research 107(1):101-7, 2002.

Soto L J, Sorenson B S, Nelson B W, Leonard A S, Saltzman D A; Generation of Immunity to Hepatic Metastases by Attenuated Salmonella typhimurium, Ann Surg Onc, 11(2)suppl: S107, 2004.

Soto L J, Sorenson B S, Kim A S, Feltis B A, Leonard A S, and Saltzman D A: Attenuated Salmonella typhimurium prevents the Establishment of Unresectable Hepatic Metastases and Improves Survival in a Murine Model. Journal of Pediatric Surgery 38(7) 1075-1079, 2003.

Barnett S J, Soto III L J, Sorenson B S, Nelson B W, Leonard A S, Saltzman D A. Salmonella typhimurium Invades and Decreases Tumor Burden in Neuroblastoma. Journal of Pediatric Surgery 40(6): 993-998, 2005.

Brent S. Sorenson, B S, Kaysie L. Banton, M D, Natalie L Frykman, B S, Robert D. Acton M D, Arnold S. Leonard M D, PHD, Daniel A. Saltzman, M D, PhD. Attenuated Salmonella typhimurium with IL-2 gene Reduces Pulmonary Metastases in a Model of Osteosarcoma; Clinical Orthopaedics and Related Research. 466(6): 1285-91. 2008.

Brent S. Sorenson, B S, Kaysie L. Banton, M D, Natalie L Frykman, B S, Robert D. Acton M D, Arnold S. Leonard M D, PHD, Daniel A. Saltzman, M D, PhD. Attenuated Salmonella typhimurium with IL-2 gene Prevents the Establishment of Pulmonary Metastases in a Model of Osteosarcoma; J Ped Surg. 43(6): 1153-8. 2008.

Drees J, Mertensotto M, Liu G, Panyam, J, Leonard A, Augustin L, Schottel J, and Saltzman D: Attenuated Salmonella enterica Typhimurium Reduces Tumor Burden in an Autochthonous Breast Cancer Model. Anticancer Research, 35(2):843-849, 2015. PMID 25667465

Drees J J, Mertensotto M J, Augustin L B, Schottel J K. Saltzman D A: Attenuated Salmonella Vascular Disruption Enhances Bacterial Targeting of Autochthonous Tumors, 2015 Journal of Cancer, 6(9):843-848. PMID 26284135

Saltzman D A, Augustin L B, Leonard A S, Mertensotto M, and Schottel J: Low Dose Chemotherapy Combined with Attenuated Salmonella Significantly Reduces Tumor Burden and is Less Toxic than High Dose Chemotherapy in an Autochthonous Murine Model of Breast Cancer. Surgery, 163(3):509-514, 2018. PMID 29229318

Fritz S E, Henson M S, Greengard E, Winter A L, Stuebner K M, Yoon U, Wilk V L, Borgatti A, Augustin L B, Modiano J F, Saltzman D A. A phase I clinical study to evaluate safety of orally administered, genetically engineered Salmonella enterica serovar Typhimurium for canine osteosarcoma. Vet Med Sci. 2016; 2(3):179-90.

Gniadek T, Augustin L, Schottel J, Leonard A, Saltzman D, Greeno E, Batist G. A Phase I, Dose Escalation, Single Dose Trial of Oral Attenuated Salmonella Typhimurium Containing Human IL-2 in Patients with Metastatic Gastrointestinal Cancers. Journal of Immunotherapy. Volume 43(7): 217-221, September 2020 doi: 10.1097/CJI.0000000000000325.PMID: 32554977

Batist G, Park J Y, Drees J, Kangas T, Saltzman D. Orally Administered Multiple Dose Saltikva (Salmonella-IL2) in Conjunction with Folfirinox in a Patient with Stage IV Pancreatic Cancer: A Case Report. Clinical Oncology: Case Reports. 2020; 3(3).

In one embodiment, the target is transforming growth factor-beta (TGF-β). TGF-β is a central mediator of fibrosis in various organs, including the liver. It promotes the activation of hepatic stellate cells (HSCs) into myofibroblasts, which are responsible for producing collagen and other matrix proteins. Inhibiting TGF-β signaling or its downstream pathways is a major approach in anti-fibrotic therapy.

In another embodiment, the target is platelet derived growth factor (PDGF). PDGF stimulates the proliferation of HSCs and contributes to their activation into fibrogenic myofibroblasts. Inhibitors targeting PDGF receptors (PDGFRs) could be utilized to reduce liver fibrosis.

In another embodiment, the target is connective tissue growth factor (CTGF). CTGF is induced by TGF-β and plays a role in collagen production and fibrosis progression. Inhibitors of CTGF expression or activity could be used as potential anti-fibrotic agents.

In another embodiment, the target is matrix metalloproteinases (MMPs). MMPs are enzymes that degrade extracellular matrix components, including collagen. In liver fibrosis, the balance between MMPs and their inhibitors (TIMPs, tissue inhibitors of metalloproteinases) is disrupted. Strategies to enhance MMP activity or inhibit TIMPs could reduce fibrosis.

In another embodiment, the target is Endothelin-1 (ET-1). ET-1 is a vasoconstrictor peptide that also promotes fibrogenesis by stimulating collagen production and HSC activation. ET-1 receptor antagonists could be used as potential anti-fibrotic therapies.

In another embodiment, the target is nuclear Factor-kappa B (NF-κB). NF-κB is a transcription factor involved in inflammation and fibrosis. Inhibiting NF-κB signaling may reduce inflammation and subsequently decrease fibrosis progression.

In another embodiment, the target is periostin. Periostin is a matricellular protein that promotes fibrosis by activating HSCs and promoting collagen deposition. Inhibiting periostin expression or activity could reduce fibrosis of the liver.

In another embodiment, what is claimed is a liver treatment agent comprising an effective amount of attenuated Salmonella typhimurium containing a plasmid carrying a coding sequence encoding a therapeutic composition, said coding sequence directed to a TGF-β inhibitor.

In another embodiment, what is claimed is a liver treatment agent comprising an effective amount of attenuated Salmonella typhimurium containing a plasmid carrying a coding sequence encoding a therapeutic composition, said coding sequence directed to a PDGF inhibitor.

In another embodiment, what is claimed is a liver treatment agent comprising an effective amount of attenuated Salmonella typhimurium containing a plasmid carrying a coding sequence encoding a therapeutic composition, said coding sequence directed to a CTGF inhibitor.

In another embodiment, what is claimed is a liver treatment agent comprising an effective amount of attenuated Salmonella typhimurium containing a plasmid carrying a coding sequence encoding a therapeutic composition, said coding sequence directed to a TIMP inhibitor.

In another embodiment, what is claimed is a liver treatment agent comprising an effective amount of attenuated Salmonella typhimurium containing a plasmid carrying a coding sequence encoding a therapeutic composition, said coding sequence directed to an ET-1 receptor antagonist.

In another embodiment, what is claimed is a liver treatment agent comprising an effective amount of attenuated Salmonella typhimurium containing a plasmid carrying a coding sequence encoding a therapeutic composition, said coding sequence directed to a NF-κB inhibitor.

In another embodiment, what is claimed is a liver treatment agent comprising an effective amount of attenuated Salmonella typhimurium containing a plasmid carrying a coding sequence encoding a therapeutic composition, said coding sequence directed to a periostin inhibitor.

### Definitions

“Attenuated” means bacteria selected or altered to greatly diminish its capacity to cause disease, but still able to retain its ability to colonize the gut associated lymphoid tissue.

“CD4+” and “CD4+ cell” mean a helper subset of T cells.

“CD8+” and “CD8+ cell” mean a cytotoxic subset of T cells.

“Coding sequence” and “coding region” are used interchangeably and refer to a polynucleotide that encodes a protein and, when placed under the control of appropriate regulatory sequences, expresses the encoded protein. The boundaries of a coding region are generally determined by a translation start codon at its 5′ end and a translation stop codon at its 3′ end.

“Gated Lymphocytes” refers to lymphocytes that have been analyzed in a fluorescent cell sorter.

“IL-2” means the protein human interleukin-2.

“NK” or “NK cell” means natural killer cell.

“Operably linked” refers to a juxtaposition wherein the components so described are in a relationship permitting them to function in their intended manner. A regulator sequence is operably linked to a coding region when it is joined in such a way that expression of the coding region is achieved under conditions compatible with the regulatory sequence.

“Regulatory Sequence” refers to a nucleotide sequence that regulates expression of a coding region to which it is operably linked. Nonlimiting examples of regulatory sequences include promoters, transcription initiation sites, translation start sites, translation stop sites and terminators.

### Example 1

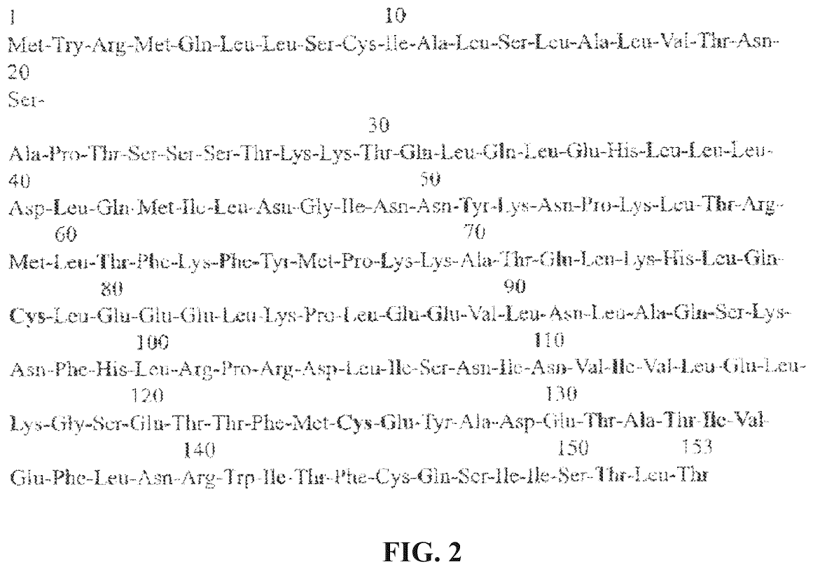

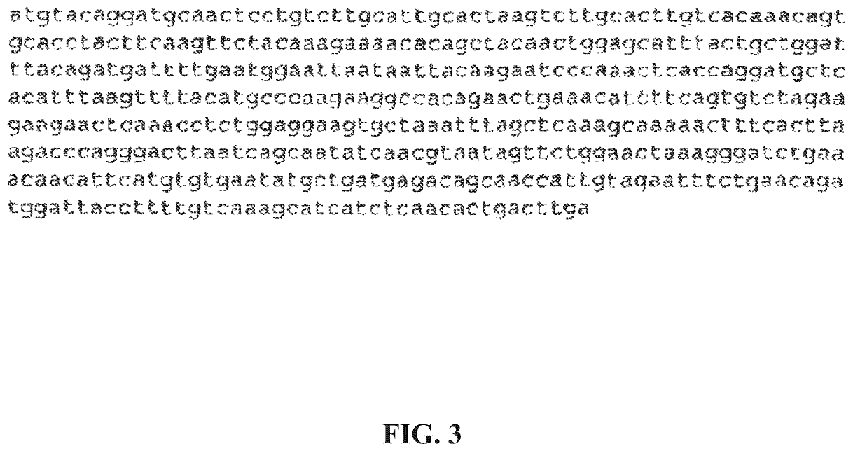

The following example relating to Interleukin-2 (IL-2) shows by way of example how proteins, nanobodies or any other encoded material can be incorporated into the methods of the present invention to promote a desired treatment for liver diseases. IL-2 (SEQ ID NO: 2-3, FIG. 2-3) is a protein naturally produced by the human body which promotes lymphocyte proliferation and enhances the cytolytic function of T cells and natural killer (NK) cells. It is thus able to stimulate the immune system to produce cancer-destroying white blood cells. IL-2 based immunotherapy in certain types of cancer has been studied for years with limited success.

Attenuated Salmonella typhimurium

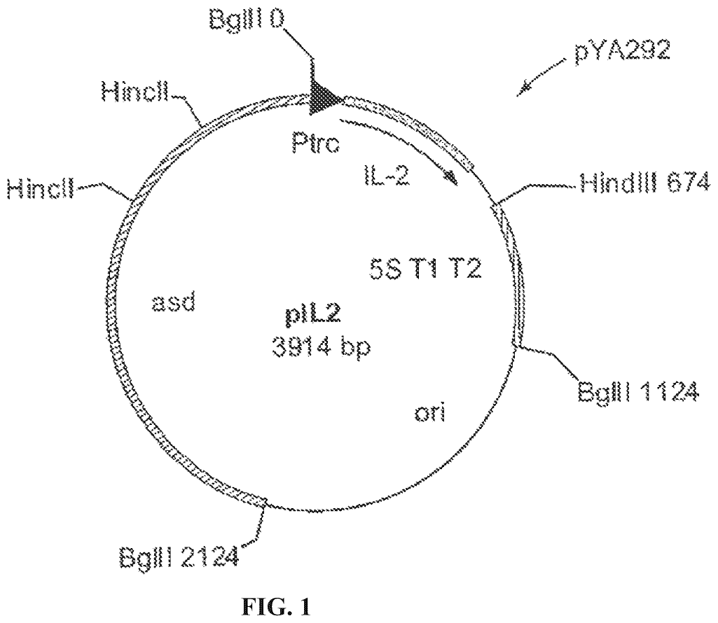

While IL-2 is naturally produced by the human body, its maximum effectiveness requires a higher concentration and more specific delivery vector to the disease site. However, high doses of IL-2 are found to result in severe toxicity in many patients. A solution to this problem was found in using a live bacterial strain of Salmonella typhimurium which was attenuated to greatly diminish its capacity to cause disease. S. typhimurium is used due to its natural ability to colonize the gut associated lymphoid tissue (GALT), liver and spleen. Colonization of the liver by the attenuated S. typhimurium further initiates a generalized cellular response against the bacteria or can persist as a carrier state. The χ4550 strain of S. typhimurium used in the present invention contains a gene deletion constructed by transposon mutagenesis with Tn10 followed by selection for furasic acid resistance. This method of genetic alteration leads to deletional loss of Tn10 and adjacent DNA sequences to produce a deletion of aspartate semialdehyde dehydrogenase (asd). This mutation imposes a requirement for diaminopimelic acid. The lack of the asd enzyme in these bacteria leads to the inability to construct a stable cell wall causing lethal lysis of the S. typhimurium. Thus, to ensure stable expression of a desired protein, a plasmid-(pYA292) (FIG. 1) was constructed which carries the asd gene. In order to ensure avirulence of the S. typhimurium strain, standard P22 phage transduction of the mouse virulent S. typhimurium SR-11 strain χ3306 was employed to construct the χ4550 strain that lacks the ability to synthesize adenylate cyclase and the CAMP receptor protein (CRP). Cyclic AMP and cAMP receptor protein are used for the transcription of many genes and operons concerned with the transport and breakdown of catabolites. Although CAMP is found in mammalian tissue and theoretically could be used by the bacteria to increase the potential for virulence, the lack of a CAMP receptor protein should abolish any benefit that could occur by the uptake of cAMP by these mutant bacteria.

A synthetic cDNA coding for a truncated human IL-2 protein, optimized for expression in Escherichia coli was inserted into plasmid pYA292 using well known methods. The truncated cDNA (SEQ ID NO: 1) is a part of the synthetic IL-2 nucleotide sequence. This sequence is one nucleotide short of the sequence that was intended to code for a full-length mature human IL-2 protein. By “mature” is meant a protein lacking the beginning (N-terminal) 20 amino acid signal sequence that is cleaved off as the molecule is secreted from a human cell. The mutation that occurred is a deletion of a “t” nucleotide between the “a” at position 272 and the “g” at position 273. This resulted in an in-frame taa stop codon at position 274 that truncated the resultant IL-2 protein. The resulting DNA nucleotide sequence is SEQ ID NO: 1 and the expressed protein is SEQ ID NO: 2. Both the aspartate semialdehyde dehydrogenase (asd+) vector and the synthetic truncated human IL-2 cDNA were digested to completion with restriction enzymes EcoRI (Promega, Madison, Wis.) and Hind III (New England Biolabs, Beverly, Mass.). The ^{˜}3.4 kb linearized vector fragment of pYA292 and the EcoRI-HindIII fragment of the IL-2 gene were isolated following agarose gel electrophoresis using the PrepaGene Kit (BioRad, Hercules, Calif.). The IL-2 gene fragment was ligated into the pYA292 vector using T4 DNA ligase (Promega, Madison, Wis.) with a 3:1 molar excess of insert and incubating for 4 hours at 16° C. The ligation mix was then electroporated into the χ4550 strain of attenuated S. typhimurium. S. typhimurium, Δcya-1 Δcrp-1 ΔasdA1 strain χ4550, was grown in Luria Broth (Sigma, St. Louis, Mo.) containing 50 mg/ml diaminopimelic acid (DAP). Cultures were grown to an absorbance of 0.200 at OD600 (approximately 10<8> colony forming units (CFU)/ml broth) and the cells were prepared for electroporation. Plasmid vector pYA292 and the ligation mix were electroporated into χ4550 utilizing an electroporation device (BioRad) with 0.2 cm disposable cuvettes. Cells were pulsed at 2.5 kV and 25 μF with a pulse controller at 200 ohms. Cells were then subsequently plated on Luria agar without DAP and recombinant clones were identified using the Magic Mini-Prep DNA Purification System (Promega), and restriction enzyme digestion with EcoRI and HindIII and gel electrophoresis with 1.2 agarose. The restriction enzyme mapping revealed a plasmid corresponding to that expected for an insert of the IL-2 fragment in pYA292 and the plasmid was renamed pIL2. The new transformant was renamed χ4550 (pIL2). Transforming an asd deleted strain with the plasmid (pIL2) allows for the stable expression of IL-2.

As discussed above, stability of this vector is maintained because the particular strain of S. typhimurium used here (χ4550) lacks the enzyme aspartate semialdehyde dehydrogenase (asd), which, conversely, the plasmid containing the IL-2 gene (pIL2) contains. Bacteria lacking asd cannot make diaminopimelic acid (DAP), an essential component of the bacterial cell wall and, thus, would not long survive. Thus, if the attenuated S. typhimurium were to attempt to revert to its wild-type strain and lose the plasmid, it would die a “DAP-less” death. Because the loss of the IL-2 containing plasmid would also result in the loss of the plasmid encoded asd, stable expression of the IL-2 gene is achieved.

It is believed that a coding sequence encoding truncated human interleukin-2 having an eighty percent identity to SEQ ID NO: 1 would be effective in the treating and preventing cancerous tumors. It is further believed that a coding sequence encoding truncated human interleukin-2 having an eighty five percent identity to SEQ ID NO: 1 would be effective in treating and preventing cancerous tumors. It is also believed that a coding sequence encoding truncated human interleukin-2 having a ninety percent identity to SEQ ID NO: 1 would be effective in treating and preventing cancerous tumors. It is further believed that a coding sequence encoding truncated human interleukin-2 having a ninety five percent identity to SEQ ID NO: 1 would be effective in treating and preventing cancerous tumors.

Murine Hepatic Metastases from Adenocarcinoma Cells.

The following paragraph is for background information only and is not intended to constitute an admission of prior art.

Experimental data was reported in Saltzman, Heise, Hasz, Zebede, Kelly, Curtiss, Leonard, and Anderson, “Attenuated Salmonella typhimurium Containing Interleukin-2 Decreases MC-38 Hepatic Metastases: a Novel Liver treatment Agent,” Cancer Biotherapy and Radiopharmaceuticals 11:2 (1996). At the time, it was believed that the plasmid pIL2 contained DNA encoding normal interleukin-2. However, it was recently discovered that the DNA in the plasmid pIL2 contained a single base pair deletion encoding a truncated protein (SEQ ID NO: 2). This was recently noted in Sorenson, Banton, Frykman, Leonard, and Saltzman, “Attenuated Salmonella typhimurium with IL-2 Gene Reduces Pulmonary Metastases in Murine Osteosarcoma,” Clin Orthop Relat Res (2008) 466:1285-1291. Neither of the above-noted publications discloses the nucleotide sequence (SEQ ID NO:1) for the truncated DNA or the amino acid sequence (SEQ ID NO: 2) for the truncated protein.

The following discussion refers to the truncated DNA and protein.

Two basic tumor models were used to examine the efficacy of this novel liver treatment system on hepatic metastases from adenocarcinoma cells: a Tumor Treatment Model and a Tumor Prevention Model.

In the Tumor Treatment Model female 6 to 8 week old C57BL/6 mice were purchased from Harlan Sprague-Dawley (Indianapolis, Ind.). At the onset of each experiment, the mice were randomly divided into four groups (Control, Salmonella typhimurium-IL-2, antioxidant oil alone and Salmonella typhimurium IL-2 with antioxidant oil) that were orally inoculated with saline or 10<8> S. typhimurium χ4550pIL2 and received a standard rodent diet or a standard rodent diet supplemented with antioxidant oil for the duration of the experiment. The procedure yielded four groups: saline, antioxidant oil, S. typhimurium χ4550pIL2, and S. typhimurium χ4550pIL2+ antioxidant oil. Mice in the antioxidant oil and S. typhimurium χ4550pIL2+ antioxidant oil groups received a standard rodent diet supplemented with black raspberry seed oil from Botanic Oil Innovation, Inc. (Spooner, Wis.) at a concentration of ten percent by weight. In order to incorporate the antioxidant oil into the diet, it was necessary to crush the standard rodent diet pellets to the consistency of coarse sand. To negate any possible variation in food consumption due to the form of the diet, all groups received a crushed diet. Mice were fed their respective diets and water ad libitum. 25,000-100,000 MCA murine adenocarcinoma cells were injected into the spleen to facilitate hepatic metastases via the portal circulation on Day 0. On Day 3 the mice were randomized into their groups and treated. On Day 12 of experimentation, mice were sacrificed and liver metastases were enumerated for number and volume of tumor.

In the Tumor Prevention Model, at the onset of each experiment, mice were administered the control (saline), antioxidant oil, S. typhimurium χ4550pIL2 with and without the antioxidant oil on Day 0. On Day 7 splenic injection of 50,000 MCA-38 adenocarcinoma cells was accomplished. On Day 14 hepatic metastases were enumerated for tumor number and volume. Total tumor volume was calculated assuming tumor shape as a sphere (4/3 r<3>). Hepatic lymphocytes were also analyzed from each experimental.

Experiments were concluded at 3, 7 or 14 days following oral inoculation and all mice were sacrificed under anesthesia. A splenectomy was performed to allow for splenic lymphocyte analysis. Splenic lymphocytes were prepared by modifying a technique used to isolate hepatic lymphocytes. Briefly, the spleen was mechanically minced, passed through 100-gauge nylon mesh (Sefar America, Inc., Kansas City, Mo.), and suspended in DMEM (Sigma, St. Louis, Mo.) with 10% fetal goat serum (Sigma). Individual specimens were place on lymphocyte separation medium (Mediatech, Inc., Herndon, Va.) and centrifuged at 300 g for 60 minutes at room temperature. The mononuclear cell layer was harvested and washed twice in phosphate buffered solution (Gibco, Grand Island, N.Y.) with centrifugation at 300 g for 10 minutes at room temperature.

Splenic lymphocytes were stained with a combination of fluorochrome-conjugated anti-mouse monoclonal antibodies, including anti-NK1.1, anti-CD4, and anti-CD8 (all obtained from BD Biosciences Pharmingen, San Diego, Calif.). Lymphocyte staining was performed at 4° C. for 30 minutes by incubating the cells with monoclonal antibodies. After washing, analysis was performed with a FACScan cytofluorometer (Becton-Dickinson, Grenoble, France) using CellQuest software (Becton-Dickinson). Viable lymphocytes were gated by side and forward scatter profiles. For each specimen, analysis was based on 10,000 acquired events.

Statistical analyses were performed using StatView 5.0 (SAS Institute, Cary, N.C.). At the conclusion of an experiment, splenic lymphocyte phenotype was analyzed by analysis of variance followed by Fisher's test for significant difference. Experiments were repeated twice on separate days to verify reproducibility. Statistical significance was regarded as P<0.05.

### Tumor Treatment Model

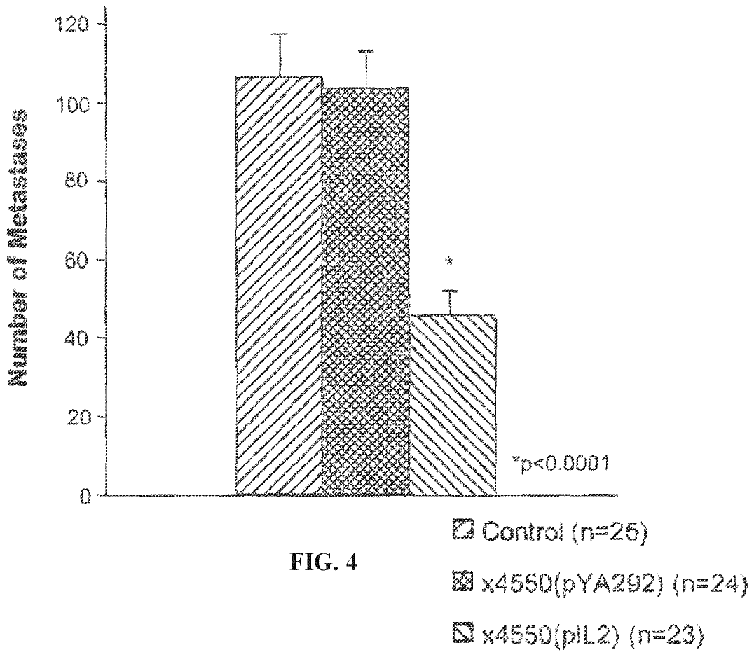

FIG. 4 shows the results of the control, S. typhimurium χ4550 and S. typhimurium χ4550pIL2 groups on hepatic metastases when administered orally to tumor burdened mice. A statistically significant decrease in hepatic colorectal metastases is shown. There was a mean of 106.4 metastases in the control group, a mean of 103.7 metastases in the group fed bacteria without the gene for truncated IL-2, and a mean of 44.3 metastases in the group fed bacteria with the gene for truncated IL-2.

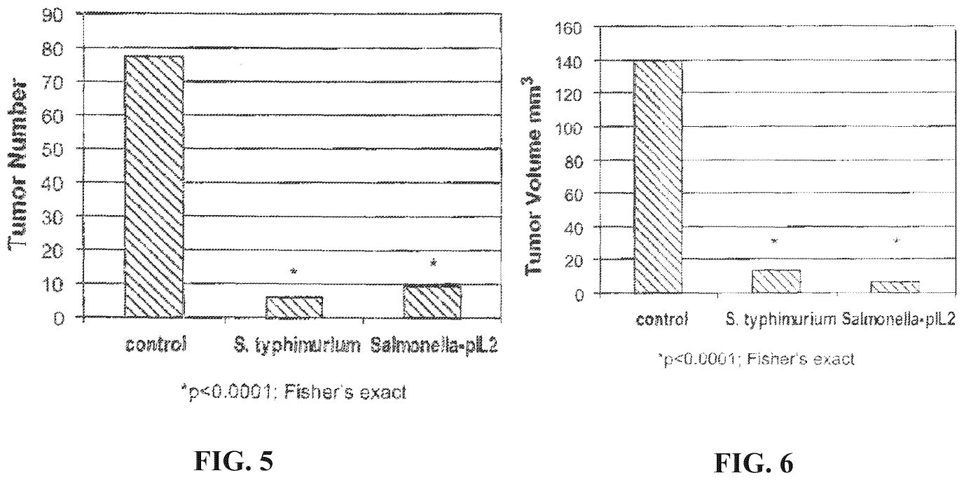

FIG. 5 shows additional results of the control, S. typhimurium χ4550 and S. typhimurium χ4550pIL2 groups on tumor number when administered orally to tumor burdened mice. A statistically significant reduction in tumor number is shown.

FIG. 6 shows the results of the control, S. typhimurium χ4550 and S. typhimurium χ4550pIL2 groups on tumor volume when administered orally to tumor burdened mice. A statistically significant reduction in tumor volume is shown.

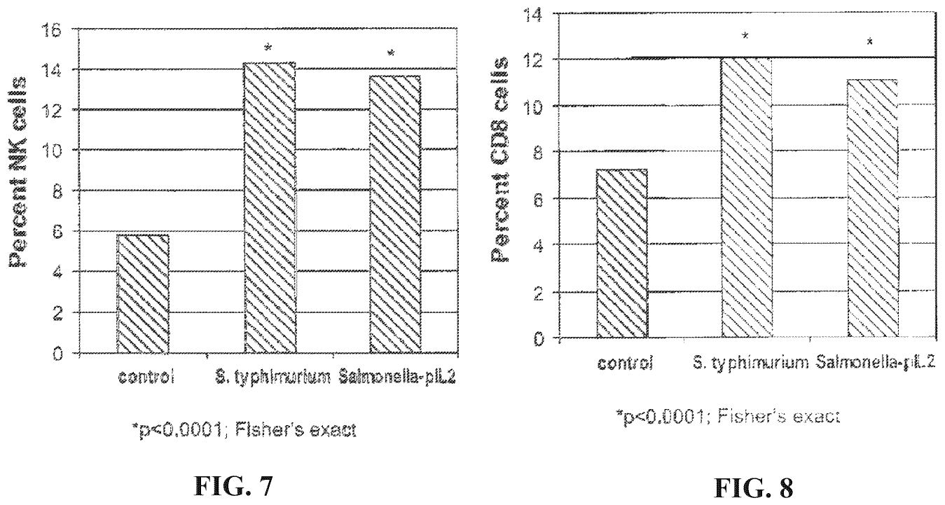

FIG. 7 shows the results of the control, S. typhimurium χ4550 and S. typhimurium χ4550pIL2 groups on NK cells when administered orally to tumor burdened mice. An increase in NK cells is shown in the S. typhimurium χ4550 and S. typhimurium χ4550pIL2 groups when compared to the control group.

FIG. 8 shows the results of the control, S. typhimurium χ4550 and S. typhimurium χ4550pIL2 groups on CD8+ cells when administered orally to tumor burdened mice. An increase in CD8+ cells is shown in the S. typhimurium χ4550 and S. typhimurium χ4550pIL2 groups when compared to the control group.

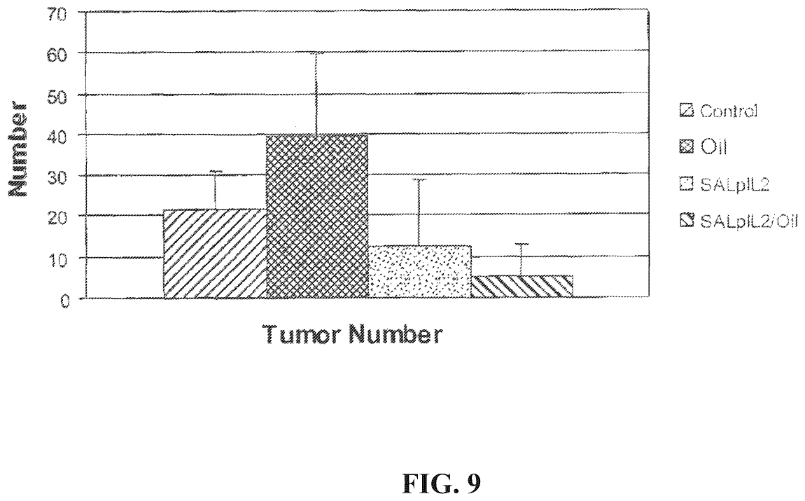

FIG. 9 shows the results of the four groups (control, oil, S. typhimurium χ4550pIL2 and S. typhimurium χ4550pIL2 plus antioxidant oil) on total tumor number in tumor burdened mice. As shown, the S. typhimurium χ4550pIL2 plus antioxidant oil group had approximately an eight-fold reduction in total number of tumor cells compared to the group receiving only oil. An approximate two-fold reduction in the number of tumors was shown compared to the group receiving only S. typhimurium χ4550pIL2. Compared to the control group, the group receiving S. typhimurium χ4550pIL2 plus antioxidant oil showed a four-fold reduction in the number of tumors.

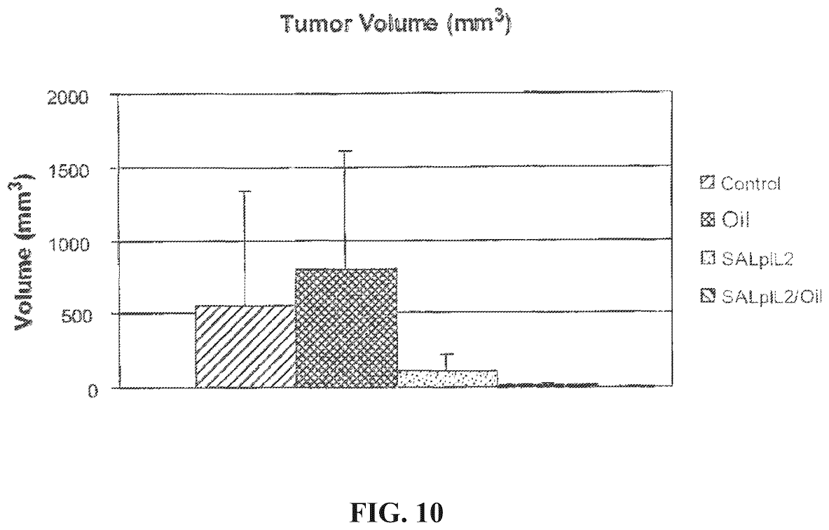

FIG. 10 shows the results of the four groups (control, antioxidant oil, S. typhimurium χ4550pIL2 and S. typhimurium χ4550pIL2+ antioxidant oil) on tumor volume in tumor burdened mice. As shown the S. typhimurium χ4550pIL2 plus antioxidant oil group showed almost negligible tumor volume compared to the antioxidant oil only group, the control group, and the S. typhimurium χ4550pIL2 only group. Tumor Prevention Model

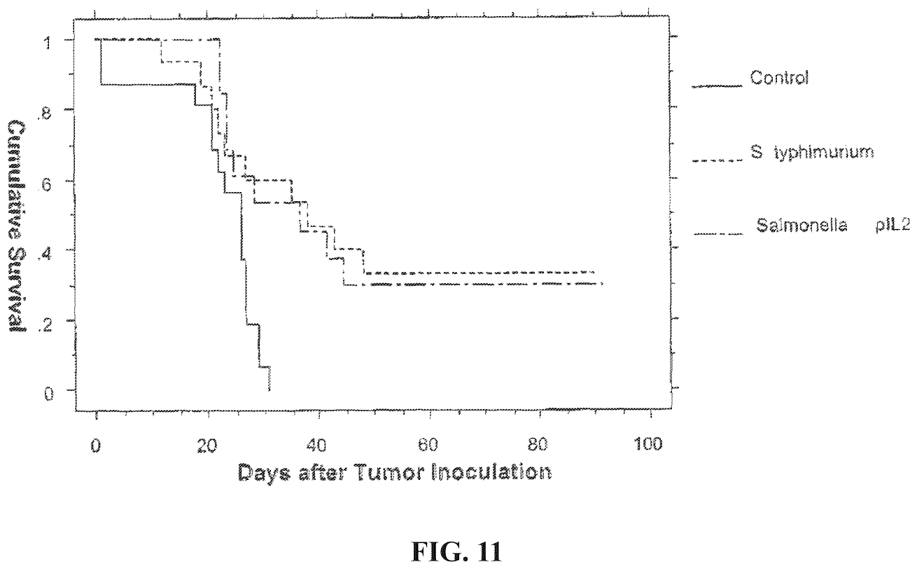

FIG. 11 shows the cumulative survival of tumor naive mice after being fed control, S. typhimurium χ4550 and S. typhimurium χ4550pIL2, The groups receiving S. typhimurium χ4550 and S. typhimurium χ4550pIL2 show an almost 40% long term survival rate over the control group.

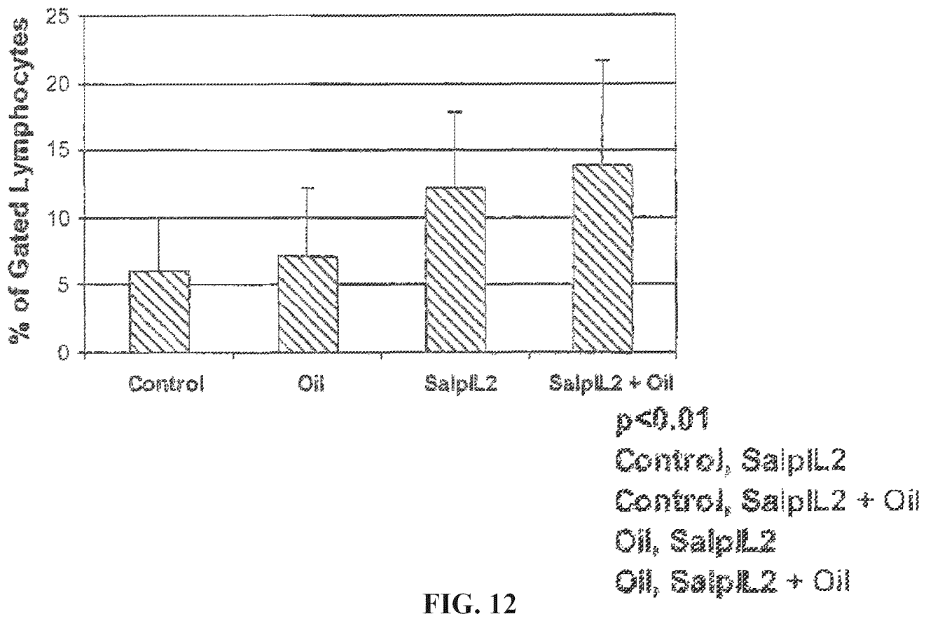

FIG. 12 shows the results of the four groups (control, antioxidant oil, S. typhimurium χ4550pIL2 and S. typhimurium χ4550pIL2 plus antioxidant oil) on NK cell population in tumor naive mice. A statistically significant increase over the control group and antioxidant oil only group is shown in the S. typhimurium χ4550pIL2 and S. typhimurium χ4550pIL2 plus antioxidant oil groups.

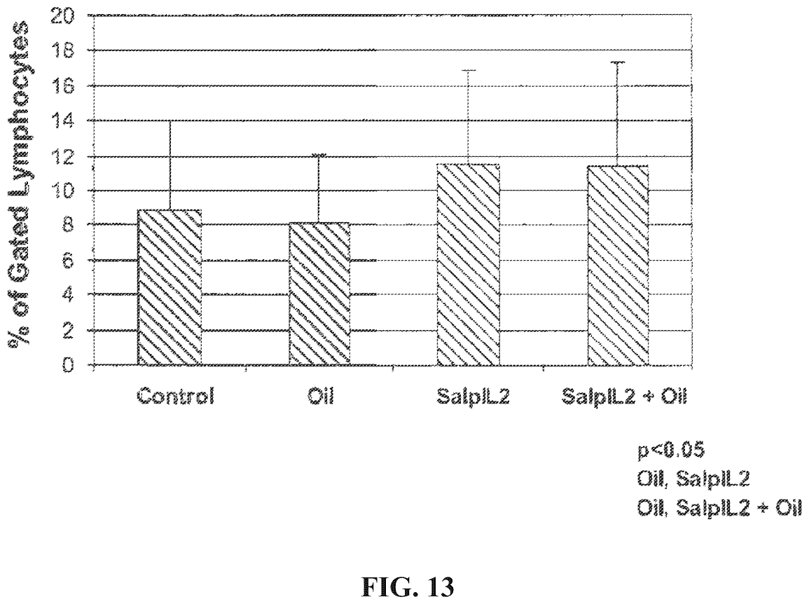

FIG. 13 shows the results of the four groups (control, antioxidant oil, S. typhimurium χ4550pIL2 and S. typhimurium χ4550pIL2 plus antioxidant oil) on CD8+ cell population in tumor naive mice. A slight increase in CD8+ T cell population is shown in the mice in the S. typhimurium χ4550pIL2 plus antioxidant oil group.

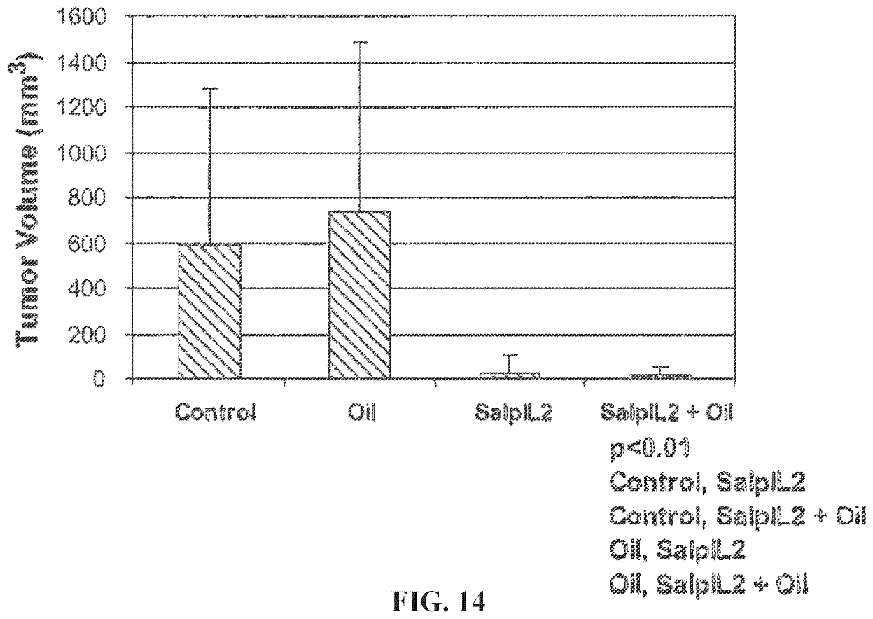

FIG. 14 shows the results of the four groups (control, antioxidant oil, S. typhimurium χ4550pIL2 and S. typhimurium χ4550pIL2 plus antioxidant oil) on CD4+ T helper cell population in tumor naive mice. An overall statistically significant increase in CD4+ T helper cell population is shown in the S. typhimurium χ4550pIL2 plus antioxidant oil group.

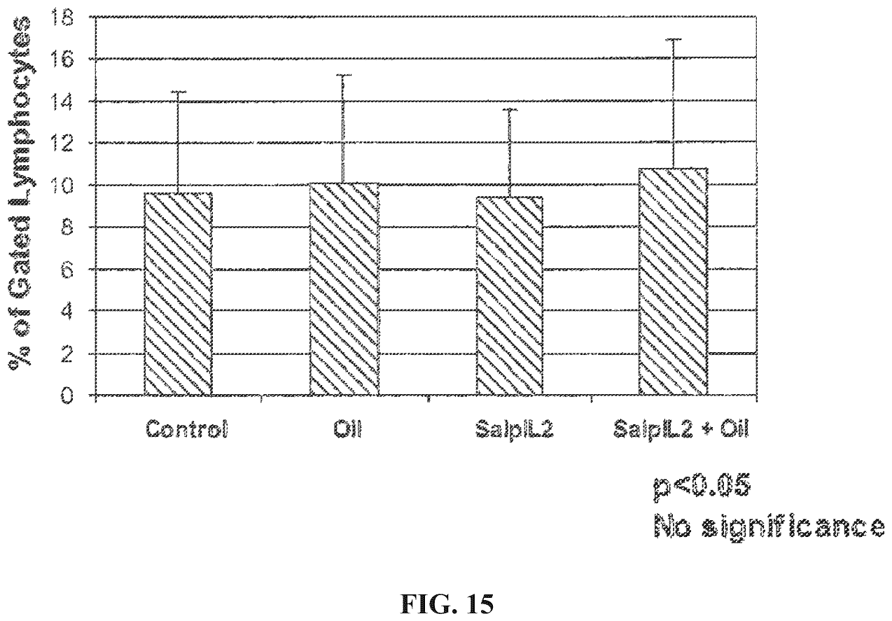

FIG. 15 shows the results of the four groups (control, antioxidant oil, S. typhimurium χ4550pIL2 and S. typhimurium χ4550pIL2 plus antioxidant oil) on tumor number in tumor naive mice. A statistically significant decrease in tumor number is shown in the S. typhimurium χ4550pIL2 and S. typhimurium χ4550pIL2 plus antioxidant oil groups.

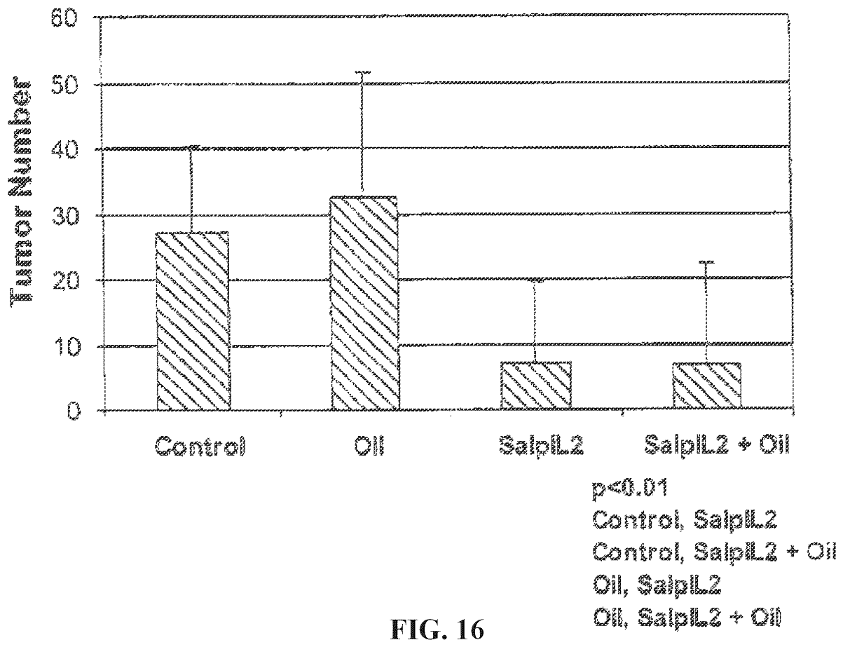

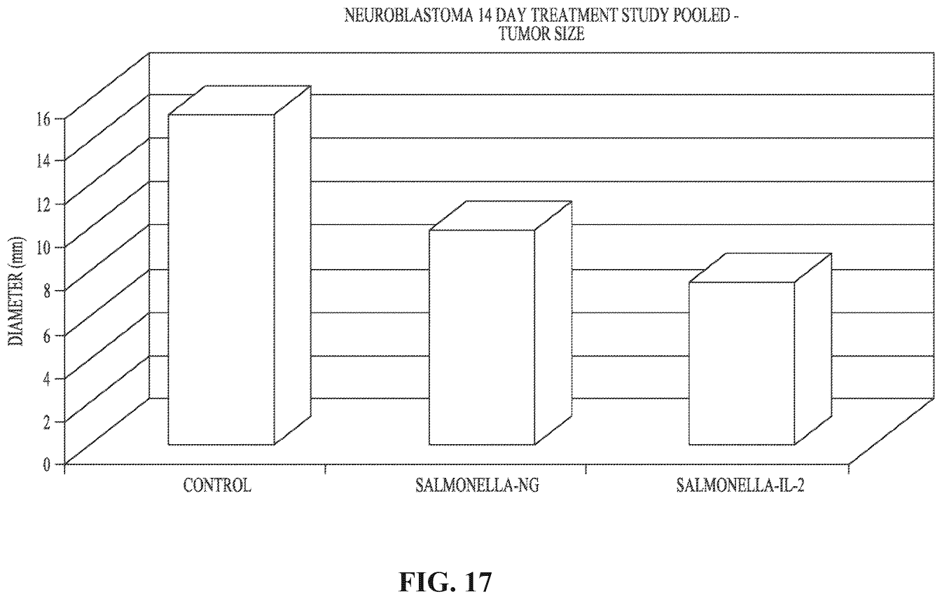

FIG. 16 shows the results of the four groups (control, antioxidant oil, S. typhimurium χ4550pIL2 and S. typhimurium χ4550pIL2 plus antioxidant oil) on tumor volume in tumor naive mice. A statistically significant decrease in tumor volume is observed in the S. typhimurium χ4550pIL2 group, with a further decrease observed in the S. typhimurium χ4550pIL2 antioxidant oil group.

### Treatment of Retroperitoneal Neuroblastoma.

The following paragraph is for background information only and is not intended to constitute an admission of prior art.

Experimental data was reported in Daniel A. Saltzman, “Cancer Immunotherapy based on the Killing of Salmonella typhimurium-infected Tumour Cells,” Expert Opin. Biol. Ther. (2005) 5(4): 443-449. At the time, it was believed that the plamid pIL2 contained DNA encoding normal interleukin-2. However, it was recently discovered that the DNA in the plasmid pIL2 was actually a single base-pair deleted DNA encoding a truncated protein (SEQ ID NO: 2). This was recently noted in Sorenson, Banton, Frykman, Leonard, and Saltzman, “Attenuated Salmonella typhimurium with IL-2 Gene Reduces Pulmonary Metastases in Murine Osteosarcoma,” Clin Orthop Relat Res (2008) 466:1285-1291. Neither of the above-noted publications discloses the nucleotide sequence (SEQ ID NO:1) for the truncated DNA or the amino acid sequence (SEQ ID NO:2) for the truncated protein.

The following data are for the truncated DNA and protein.

Neuroblastoma is the most common malignancy in infants and the most common extracranial solid tumor of childhood. Infants and children with confined disease have a >80% chance of cure, whereas older children with metastatic disease have only a 20-25% cure rate. Although neuroblastoma accounts for only 10% of all childhood tumors, it is responsible for 15% of all cancer-related deaths in the pediatric age group. Multiple therapies are being studied to treat these children with state IV disease, including radioactive iodine-131-meta-iodobenzylguanidine (MIBG) therapy, antiobiotics directed at the ganglioside GD2, and 13-cis-retinoic acid therapy combined with chemotherapy. These therapies have shown inconsistent response rates towards treatment, and ongoing studies continue to delve into their utility.

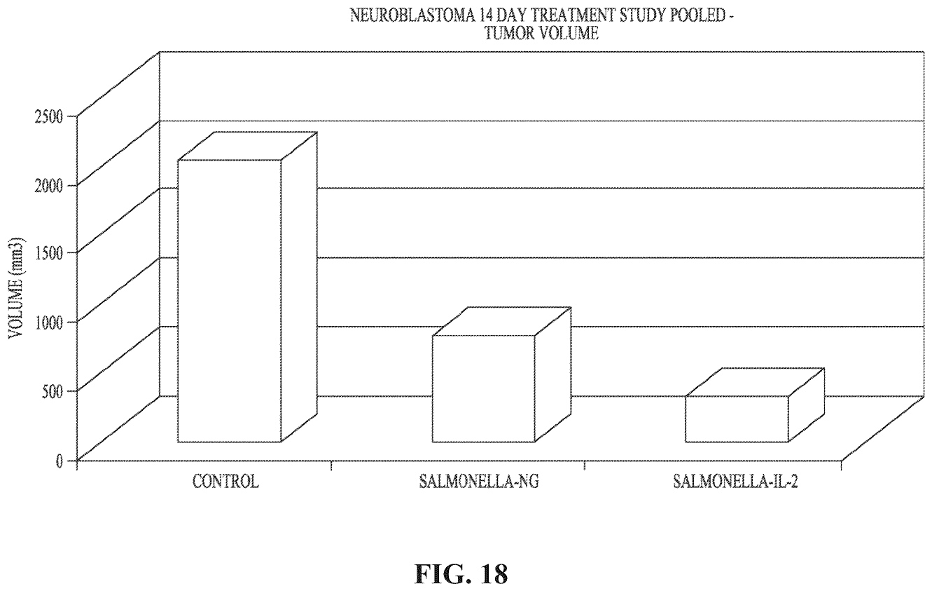

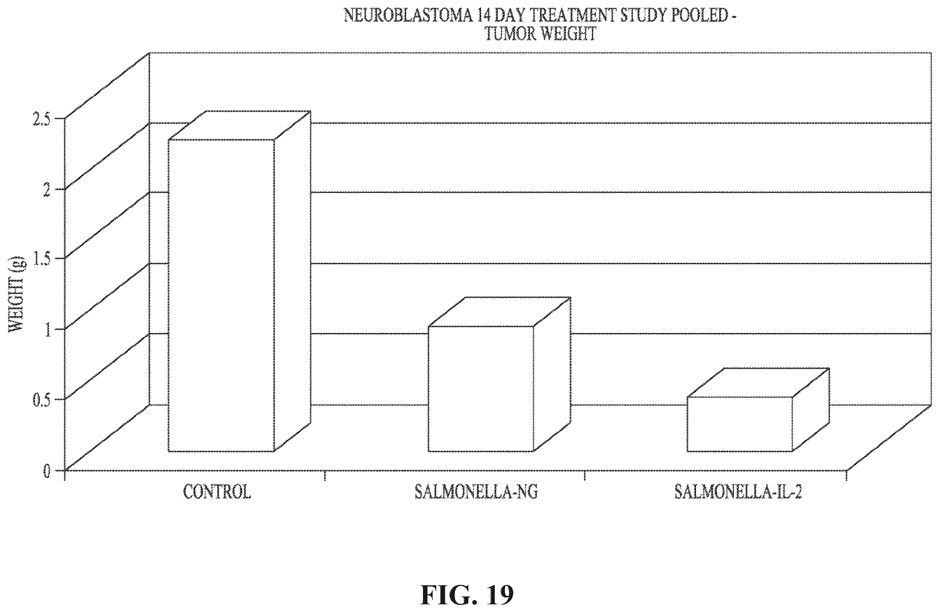

Due to the finding of an increase of Salmonella invasion and division efficiency in neuroblastoma cells, the antitumor effect of Salmonella in a preclinical murine model of a retroperitoneal neuroblastoma was studied. In these experiments, it was found that treatment with S. typhimurium or Salmonella-pIL2 in those mice with retroperitoneal neuroblastomata resulted in a significant reduction in tumor volume (749.5 and 332.4 mm3, respectively) when compared with saline controls (2024.3 mm3; p<0.00001) (FIG. 18). Treatment with S. typhimurium or Salmonella-pIL2 is also associated with a reduction in tumor weight (0.88 and 0.377 grams, respectively) when compared with saline controls (2.218 grams; p<0.0001) (FIG. 19). When comparing reductions between S. typhimurium and Salmonella-pIL2 treated animals, there is a decrease in tumor volume (749.5 and 332.4 mm3; p<0.0001) and in tumor weight (0.88 and 0.377 grams; p<0.0001), which are both statistically significant. Treatment with S. typhimurium and Salmonella-pIL2 results in a reduction in tumor burden (63 and 84%, respectively) in animals with retroperitoneal neuroblastoma.

### Prevention and Treatment of Pulmonary Metastases in Murine Osteosarcoma.

Brent S. Sorenson, Kaysie L. Banton, Natalie L. Frykman, Arnold S. Leonard, Daniel A. Saltzman, “Attenuated Salmonella typhimurium with Interleukin 2 Gene Prevents the Establishment of Pulmonary Metastases in a Model of Osteosarcoma,” Journal of Pediatric Surgery (2008) 43, 1153-1158; “Attenuated Salmonella typhimurium with IL-2 Gene Reduces Pulmonary Metastases in Murine Osteosarcoma,” Clin Orthop Relat Res (2008) 466:1285-1291.

Osteosarcoma is the most common primary bone cancer, with approximately 900 new cases annually in the United States. There is a peak incidence in early adolescence correlated with pubertal bone growth and a second peak after age 50. Primary tumors develop in the distal femur and proximal tibia and humerus. Current management of primary osteosarcoma involves surgical resection with wide margins or limb amputation in conjunction with pre- and postoperative neoadjuvant chemotherapy. Survival from local disease has improved from 20% in 1970 to approximately 70% at 3 years with the advent of current treatment with high-dose methotrexate, cisplatin, ifosfamide, and doxorubicin. Despite the dramatic enhancement in patients' event free survival, toxicity affects nearly all patients treated with these therapies. However, in patients who present with metastatic disease detectable by CT, less than 30% disease-free survival has been achieved. In some cases, intravenous interleukin-2 treatment has resulted in complete regression of the primary tumor, though severe side effects have been noted, including fever, nausea, capillary leak syndrome, and death.

A single oral dose of an attenuated Salmonella typhimurium genetically engineered with a gene for a truncated human interleukin-2 (SalpIL2) was demonstrated to substantially reduce unresectable adenocarcinoma metastases to the liver in experimental treatment and prophylactic mouse models. In addition, SalpIL2 reduces the volume and mass of retroperitoneal neuroblastoma tumors in an experimental murine treatment model. Interestingly, the Salmonella species of bacteria also have a unique propensity to colonize tumor cells. In vitro experiments have demonstrated the ability of SalpIL2 to invade and divide preferentially within K7M2 osteosarcoma cells with respect to primary murine hepatocytes. Thus SalpIL2 may be able to persist for long periods in malignant tissues providing a prolonged antigen presentation state and enhanced immune response in the region.

Based on previous observations, SalpIL2 would substantially reduce osteosarcoma pulmonary metastases by increasing splenic and local NK cell populations in this newly developed experimental model.

Reduction of Pulmonary Metastases after Injection with Murine Osteosarcoma Cells.

In triplicate experiments, 45 balb/c mice were administered murine K7M2 osteosarcoma cells by tail vein injection. Three days later, animals were orally given saline or attenuated Salmonella species; they were then euthanized on day 21 for tumor enumeration, volume, and assessment of systemic NK and T cell populations. In an additional experiment, animals were harvested for pulmonary lymphocyte analysis.

Attenuated S. typhimurium v4550 and plasmid pYA292 were a gift from Dr. Roy Curtiss III, Washington University, St. Louis, Mo. v4550 was attenuated by Tn10 transposon mutagenesis to remove adenylate cyclase (cya), cyclin adenosine monophosphate receptor protein (crp), and aspartate semialdehyde dehydrogenase (asd) genes from the bacterial genome. These mutants have virulence factors removed, but retain immunogenic properties. Plasmid constructs with and without the truncated gene for human interleukin-2 (SalpIL2) were electroporated into v4550 using well-described techniques and renamed SalpIL2 and Sal-NG [28]. Standardized glycerol stocks of approximately 10<8>CFU/mL were prepared by creating growth curves for overnight cultures in Luria broth (Difco Laboratories, Detroit, Mich.) and freezing aliquots with an O.D.600 of 0.160 in liquid nitrogen. For experiments, cryovials were thawed to room temperature, serially diluted, and plated on MacConkey agar plates to verify CFU concentration. Use of S. typhimurium with a gene for a truncated human interleukin-2 was approved by the University of Minnesota Institutional Biosafety Committee (numbers 541 and 542).

Female balb/c mice 6 to 8 weeks old were acquired from Harlan Sprague Dawley (Indianapolis, Ind.) and housed in microisolator cages, fed standard mouse chow and water ad libitum, and given 12 hours light/dark cycles under the strict care of the University of Minnesota Research Animal Resources.

The murine osteosarcoma cell line K7M2 was acquired from the American Type Culture Collection and maintained in 25 mL DMEM, 10% fetal bovine serum, 1% penicillin, streptomycin, and L-glutamine (Sigma Chemical, St. Louis, Mo.) at 37° C. at 5% CO2. Media was changed twice weekly and cells were not allowed to become confluent. Tumor cells were incubated with 0.3% trypsin EDTA (Invitrogen, Carlsbad, Calif.) at 37° C. at 5% CO₂ for 3 minutes or until nonadherent. Cells were serially washed in Hanks' balanced salt solution (HBSS, Invitrogen) before enumeration via trypan blue exclusion (Sigma Chemical) on a phase contrast hemocytometer (Hausser Scientific, Horsham, Pa.). The suspension was diluted to a concentration of 2×10<6> cells per mL and placed on ice prior to injection. All tumor preparations were more than 90% viable and used within 1 hour of preparation.

A model was developed for pulmonary metastases for these experiments. Similar techniques have been implemented for quantifying the metastatic potential of the K7M2 cell line. In triplicate experiments, animals were anesthetized by intraperitoneal injection of 2:1 xylazine 20 mg/ml (Phoenix Pharmaceuticals, St. Joseph, Mo.) and ketamine 100 mg/ml (Abbot Laboratories, North Chicago, Ill.). The animals' eyes were swabbed with Betadine ophthalmic eye ointment (Purdue Pharma LP, Stamford, Conn.) and the animals were placed in a Broome odent Holder (Kent Scientific, Torrington, Conn.). Tails were incubated for one minute in 47° C. Betadine solution (Purdue Pharma LP) to allow for vasodilatation of the left lateral tail vein and scrubbed with a 70% ethanol swab before 200,000 K7M2 cells were injected into the left lateral tail vein. Mice were placed at random in cages with microisolators and placed on a warming pad for 2 hours or until animals were walking. On Day 3, mice were orally gavaged with their respective treatments (n=5), 200 μL HBSS for controls or 3×10<7>CFU of either Sal-NG or SalpfL2. In all experiments the mice were evaluated for presence of metastases 3 weeks after injection by euthanasia followed by an intratracheal injection of 1.5 mL of 15% India-ink solution via a blunt-ended needle. The stained lungs were carefully resected and rinsed in Fekete's solution (300 mL 70% ethanol, 30 mL 37% formaldehyde, 5 mL glacial acetic acid) and then placed in fresh Fekete's solution overnight in a 60×15 mm tissue culture dish. Tumors were enumerated, their diameters were measured and volume was calculated by 4/3πr<3>, assuming the metastases were spherical. Spleens were aseptically removed and placed in 60×15 mm culture dishes for FACS analysis of splenic lymphocytes. Due to inability to collect pulmonary lymphocytes or perform histopathological analysis from Fekete stained lungs, two additional experiments with 25 mice were conducted. Lungs for histopathological analysis were aseptically removed and placed in 10% formalin and sent to the University of Minnesota's Histopathological Core for slide preparation.

Spleens and lungs for FACS analysis were incubated with 37° C. DMEM containing 10% fetal goat serum and crushed with sterile glass stoppers. Homogenates were filtered through a 150-1 m nitex mesh (Sefar American, Kansas City, Mo.) and transferred onto 5-mL lymphocyte separation medium (Ficol, Mediatech Inc., Hendon, Va.). The cell suspension was centrifuged for 1 hour and the lymphocyte layer was carefully collected. Cells were serially washed with PBS with 1% bovine serum albumin (BSA) and 0.1% NaN3 (Sigma Chemical) and split for monoclonal antibody staining. Cells were stained with DX5/CD 49a PE and CD 3 FITC for NK cell analysis and CD 8 PerCp and CD4 FITC (Phaminogen, San Diego, Calif.) for T lymphocyte populations. Cells were cold incubated for 30 minutes at 4° C. before a final wash with PSB/BSA/NaN3 and stored under foil at 4° C. until FACS analysis. Splenic and pulmonary lymphocytes collected from experimental mice were analyzed with a FACScalibur (Becton Dickenson, Grenoble, France) and analyzed with Cell Quest Pro Software (Becton Dickenson, San Jose, Calif.). Lymphocyte populations were identified using forward-scatter versus side-scatter profiles and gated for mononuclear lymphocytes. Natural killer cell populations then were identified by DX5/CD 49b<+>/Cd 3<−>, TH and TC cells by single positive populations based on 10,000 gated events.

Number of tumors, volume, and lymphocyte populations were entered for each mouse at the experimental endpoint to calculate the total mean values for each treatment group. All differences between two groups were determined by Fisher's exact test. Graphs were constructed using Micro-soft Excel (Microsoft, Redmond, Wash.). Statistical tests were performed using StatView software v. 5.0.1 (SAS Institute, Cary, N.C.)

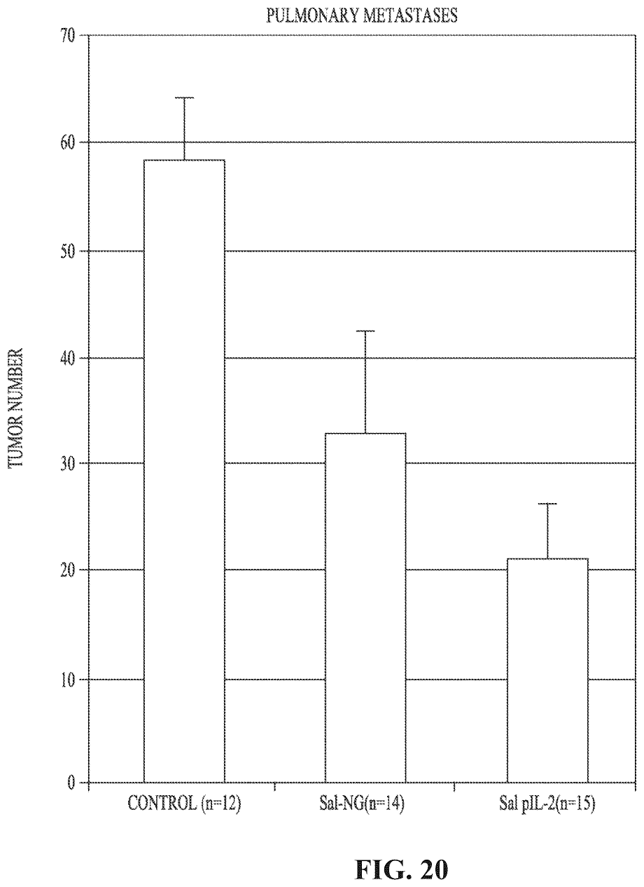

Attenuated S. typhimurium with and without a gene for truncated human interleukin-2 (SEQ ID NO: 1) had fewer total tumors (20.93 and 33, respectively; p<0.0175 and 0.0006, respectively) compared to saline controls (58.42) (FIG. 20). SalpIL2 reduced (p<0.0037) overall volume of pulmonary metastatic nodules by 78% with respect to saline controls. There was no discernable difference in the reduction of tumor number and volume between the two Salmonella treatments. NK cell populations increased (p<0.0163 and p<0.0407, respectively) in Sal-NG- and SalpIL2-treated groups (18.5% and 16.8%, respectively) with respect to saline controls (8.6). Cytotoxic T lymphocyte populations were not noticeably affected by oral administration of Sal-NG and SalpIL2 (p=0.270 and p=0.237) compared to saline controls. T helper cell populations were reduced in the SalpIL2 group (14.3%; p<0.0077) compared to saline controls (20.7%). Local pulmonary lymphocytes collected were elevated in SalpIL2 compared to control and Sal-NG treated animals (p<0.0196 and p<0.0070 respectively). Gross examination of the harvested pulmonary tissues demonstrate the reduction in the mean number of metastatic tumors by SalpIL2 with respect to saline controls. Histological analysis of the tissues treated with SalpIL2 show a decreased invasion of the metastases into the subpleural space and an increase of mononuclear cells in the area.

Prevention of Pulmonary Metastases by Administration of SalpIL2 Before Injection with Murine Osteosarcoma Cells.

Seven days before tumor injection, mice were orally gavaged with their respective treatments (n=5), 200 μL Hanks' balanced salt solution for controls or 3×10⁷ CFU of either Sal-NG or SalpIL2. Previously, it was established that a treatment model for pulmonary metastases to examine the antitumor mechanisms of SalpIL2. In triplicate experiments, animals were anesthetized by intraperitoneal injection of 2:1 xylazine, 20 mg/ml (Phoenix Pharmaceuticals, St Joseph, Mo.), and ketamine, 100 mg/ml (Abbot Laboratories, North Chicago, Ill.). On day 0, animals were prepared and administered 2×105 K7M2 mouse OS cells using well-described techniques. Mice were selected at random from their respective groups for IV tail vein injection. In all experiments, the mice were evaluated for presence of metastases 3 weeks postinjection by euthanasia followed by an intratracheal injection of 1.5 mL of 15% India ink solution via a blunt-ended needle. The stained lungs were carefully resected and rinsed in Feket's solution overnight. Tumors were enumerated and classified by the diameter of the nodules; volume was calculated by 4/3πr<3>, assuming the metastases were a sphere. Spleens were aseptically removed and placed in 60×15-mm culture dishes for fluorescent-activated cell sorting (FACS) analysis of splenic lymphocytes.

### Splenic Lymphocyte Preparation

Splenic lymphocytes were isolated by mechanically mincing spleens in DMEM containing 10% fetal goat serum. Splenic homogenates were filtered through a 150-μm nitex mesh (Sefar American, Kansas City, Mo.) and separated using density gradient centrifugation in lymphocyte separation medium (Ficol, Mediatech Inc, Hendon, Va.). Cells were serially washed with phosphate buffered saline (PBS) with 1% bovine serum albumin.

### Fluorescent-Activated Cell Sorting

Splenic lymphocytes collected from experimental mice were analyzed with a FACSCalibur (Becton Dickenson, Grenoble, France) and analyzed with Cell Quest Pro software (Becton Dickenson, San Jose, Calif.). Lymphocyte populations were identified using forward scatter vs side scatter profiles and gated for mononuclear lymphocytes. Natural killer cell populations then were identified by DX5/CD 49b<+>/Cd 3<−>, TH, and TC cells by single positive populations based on 10,000 gated events.

### Statistical Analysis

Data for tumor number, volume, and splenic lymphocytes were evaluated by analysis of variance and Fisher's Exact test using Statview statistical analysis software V 5.0.1 (SAS Institute, Cary, N.C.). Graphs and charts were constructed using Microsoft Excel (Microsoft, Redmond, Wash.).

Saline control and Salpl12 treated tissues grossly determined to be representative samples of the mean number of tumors present per group were photographed. Pretreatment with attenuated SalpIL2 and Sal-NG significantly reduced the number of pulmonary metastases (42.87 and 62.69, respectively) with respect to saline controls (183.82; P<0.0001), representing a 77% reduction in tumor. Tumor volume was reduced to 49.19 mm<3> and 58.13 mm<3> by SalpIL2 and Sal-NG as compared to 318.02 mm<3> in saline controls (P<0.0001). Furthermore, splenic NK cell populations were increased 396% with SalpIL-2 (11.25%; P<0.0007) and 426% with Sal-NG (12.76%; P<0.0003) compared to saline treated groups (2.84%). CD 8+T lymphocyte populations were unaffected by prophylaxis with SalpIL2 and Sal-NG. However, CD4+ T cells were significantly decreased at the time of animal sacrifice in SalpIL2-treated and Sal-NG-treated groups (11.58 and 11.46, respectively) as compared to saline controls (14.80; P<0.0193 and P<0.0187, respectively).

Salmonella typhimurium is a facultative intracellular organism that preferentially tracks to and divides in OS in vitro and reduces tumor burden in a prophylactic model of OS pulmonary metastasis. SalpIL2 was designed to produce a synthetic truncated IL-2, natively a 15-kDa cytokine produced by activated CD4+ T cells as well as other immune cells and is involved in the activation and proliferation of NK cells and T lymphocytes. The IV administration of IL-2 has been associated with severe side effects including fever, fatigue, malaise, and capillary leak syndrome. Numerous attempts have been made to limit the dose-dependent toxicity of IL-2 by altering the route, method, and frequency of dosing. Incorporation of IL-2 into liposomes, single or multilayered lipid vesicles, has resulted in a time-release of soluble effector compounds effecting the drug's toxicity and efficacy. In one clinical study, a liposomal preparation of IL-2 reduced IL-2 toxicity compared to soluble IL-2. In preclinical trials, liposomal IL-2 was shown to be effective in reducing pulmonary OS metastases in canines. There was an attempt to establish a local delivery system for IL-2 that may diminish the common side effects associated with IV delivery, by genetically engineering an attenuated strain of S. typhimurium to act as a biologic vector for a truncated human IL-2 gene. Other investigators have attempted the use of other strains of attenuated S. typhimurium for antitumor therapy, with limited success when given intravenously. Oral administration of SalpIL2 allows the bacteria to follow the natural route of infection and colonize various tissues including the Peyer's patches, liver, lung, and spleen.

Prophylactic models were developed based on the NK cell response after oral administration of SalpIL2. The peak NK cell response on day 14 postgavage; thus, tumor cells implanting on day 7 will encounter the highest rate of NK cell proliferation during the infection of SalpIL2. In the prophylactic model, a more dramatic reduction in tumor volume was observed than in the previous treatment model of metastatic OS. Tumor volume was reduced more than 6-fold with SalpIL2 prophylaxis with respect to saline controls. This model represents the subset of patients who do not present with radiologic detectable pulmonary metastases; however, because 80% of patients develop metastatic disease, all patients are thought to have some degree of micrometastases at time of diagnosis. Prophylaxis with SalpIL2 in the model increases systemic NK cells nearly 400% compared to saline-treated animals. Interestingly, there was no detection of a significant difference between either of the Salmonella strains. Previously, it was reported reported that a single oral dose of SalpIL2 significantly increased pulmonary and splenic NK cells in a murine model of OS. In both treatment and prevention models, there was no detection of significant differences in splenic NK or CD 8+ T-cell populations but there was a significant increase of local NK cells in the SalpIL2 group with respect to saline and Sal-NG in the treatment model animals. The presence of an increased local NK cell population suggests functionality of the recombinant IL-2 locally within the pulmonary tissue; however, the fate of the recombinant IL-2 is currently unknown. Additional experiments are necessary to investigate the location of the recombinant IL-2 and whether acute and chronic infections with SalpIL2 produce IL-2 associated toxicities.

Tumors were injected during a peak of an immune response to bacterial infection resulting in a more circulating activated NK cells, prohibiting OS tumor implantation into pulmonary tissue and enhancing tumor surveillance. Immune tumor surveillance is one theory of tumor development; tumors are constantly originating while circulating immune cells, primarily NK cells and cytotoxic T cells provide protection against malignant disease. Depression of the immune system therefore allows tumor cells the ability to escape surveillance and progress to clinical disease. Increasing NK cells may lead to increased interaction between NK and tumor cells, thus inhibiting of cellular invasion and implantation into distant sites. Tumors that do establish as pulmonary metastases are subjected to local inflammatory response to SalpIL2.

Survival for OS has not progressed since the implementation of adjuvant chemotherapy for primary tumors nearly 4 decades ago. Combination therapy has not been shown to be effective in prevention of OS pulmonary metastases. Although the 5-year survival for patients who present without pulmonary metastases has reached 70%, there has been little benefit in the long-term survival of patients who present with pulmonary metastases. Attenuated S. typhimurium strains may be a viable biological delivery vector for the treatment and prevention of metastatic OS.

While the above description of use of the present invention is specifically directed to human beings, it is also speculated that the present invention would also be effective in treating agricultural (e.g., cattle, swine, sheep, horses, domesticated fowl) and companion (dogs, cats, birds) animals.

Although the present invention has been described in considerable detail with reference to certain preferred versions thereof, other versions are also possible. Therefore, the spirit and scope of the appended claims should not be limited to the description of the preferred versions contained herein.

Salmonella are gram-negative facultative intracellular organisms that can cause a wide spectrum of disease in both humans and animals. After oral administration, Salmonella penetrate into the Peyer's patches of the intestine, where they can be phagocytosed by the resident macrophages and then carried through the reticular endothelial system before finding a safe site within the liver and spleen. In addition, Salmonella may be disseminated systemically within the local macrophages, which subsequently induce humoral and cellular immune responses. An alternative mechanism of invasion has been described where Salmonella are engulfed by dendritic cells at the mucosal surface and then transported from the gastrointestinal tract to the bloodstream by macrophages. Unlike other invasive bacteria, Salmonella can reside and divide within macrophages, the antigen-presenting cell. Peptides resulting from bacterial degradation are packaged, bound to the major histocompatibility complex (MHC) class II molecules, and presented to CD4+ T cells. In addition, antigen-specific cytotoxic CD8+ T cells against proteins expressed by the Salmonella carriers can also be stimulated. This cytotoxic response appears to take place through an alternative MHC class I processing pathway.

Capitalizing on the unique ability of this organism to invade host tissue and trigger a local as well as systemic immune response, researchers theorized that attenuation of Salmonella could preserve their abilities of invasion and immune stimulation while ameliorating their deleterious effects. Attenuation of Salmonella has been achieved by several methods. In human trials, chemical attenuation of Salmonella typhi conferred both local and systemic humoral and cellular immunity with a significant but incomplete protection against typhoid fever. Subsequently, several attenuated strains of both S. typhi and S. typhimurium were developed by deleting 2 genes encoding essential enzymes in the biosynthesis of aromatic compounds (aroA) and adenine (purA). These attenuated bacteria were well-tolerated but not as immunogenic, leading to the developments of these auxotrophic strains. Although auxotrophic strains were more immunogenic, transient bacteremia was seen in human volunteers. Curtiss et al. were primarily responsible for developing attenuated strains of Salmonella that lack the genes encoding the adenylate cyclase (cya) and cyclic adenylate cyclase (crp) receptors. These bacteria were found to be highly immunogenic and safe in human volunteers, with 80% to 100% of whom demonstrated seroconversion to bacterial antigens.

Attenuated Salmonella species are effective vaccine carriers. Examples include attenuated S. typhimurium vaccines for murine listeriosis and the human papillomavirus type 16. In addition, S. typhimurium species appear to have the unique propensity to track to tumor cells, and clinical trials have been initiated using an intravenous injection of an attenuated form of S. typhimurium, lacking aroC, in human patients with widespread malignancies. In animal studies, attenuated Salmonella as anti-cancer agents have been primarily used in mouse models, using various routes of delivery: oral or injected, either intratumoral or intravenous. One study focused on the antitumor potential in mice with intravenous injection of attenuated Salmonella to treat a multitude of tumor types that were subcutaneously implanted: Salmonella accumulated in tumors more readily than in the normal liver reservoir; despite partial inhibition of tumor growth, ultimately progressive growth was observed for all tumor types.

Characterization of the Salmonella pathogenicity island (SPI) genes revealed that one cluster, the SPI-1 genes, promote invasion from the intestine, while another cluster, the SPI-2 genes, support systemic survival within macrophages and other cells. In a subcutaneous melanoma model, SPI-2 genes appeared to be essential for tumor growth suppression.

There have been numerous studies examining the immune effectiveness of Salmonella vaccines for various entities in human volunteers. The results have been mixed—some studies documented an immune response to a particular foreign antigen, while others did not. Most researchers speculate that this mixed response results from the method of attenuation for the particular strain of Salmonella used, the type of adjuvant used, the foreign protein being expressed, or the route of administration of the Salmonella. Clinical experience with Salmonella to treat malignancies has been sparse. To date, 2 clinical studies have used Salmonella for cancer treatment. The first involved patients with metastatic malignant melanoma and metastatic renal cell carcinoma. These patients received intravenous attenuated Salmonella on a dose escalation schedule of 10⁶ to 10⁹ bacteria administered over a 4-hour interval. They all appeared to tolerate intravenous infusion of the bacteria, and some of them had tumor colonization by the bacteria, but only 1 patient demonstrated a complete response and was free of tumor 3 months after infusion. The remaining patients all had progression of disease. Interestingly, most patients demonstrated a dose-related increase of the proinflammatory cytokines interleukin-1 (IL-1), tumor necrosis factor-alpha (TNF-α), interleukin-6 (IL-6), and interleukin-12 (IL-12). In addition, most of these patients also developed strain-specific IgG and IgM antibodies.

In contrast to intravenously administered Salmonella, the second trial used an intratumoral injection of attenuated Salmonella expressing the Escherichia coli cytosine deaminase gene with concomitantly administered 5-fluorocytosine. This intratumoral study included 3 patients with various tumor types. All 3 converted 5-fluorocytosine to 5-fluorouracil, yet disease progression persisted; 1 patient had to be removed from the study because of the development of significant toxicities.

Attenuated Salmonella typhimurium Containing the Gene for Human IL-2 (Salmonella χ4550 (pIL-2))

In contrast to the Salmonella used in the referenced clinical trials, another embodiment is oral administration of the bacteria. The use of live attenuated Salmonella as IL-2 carriers has been investigated for several reasons. First, Salmonella naturally colonizes the gut-associated lymphoid tissue (GALT), liver, and spleen. Second, colonization of the liver and spleen initiates a generalized cellular immune response against the bacteria and can also induce a carrier state. In addition, attenuated Salmonella have been shown to preferentially invade various subcutaneous tumors more readily than liver tissue, at tumor-to-liver ratios ranging from 250:1 to 9000:1.

The χ4550 strain of S. typhimurium was used as the parental strain. It lacks the enzyme aspartate semialdehyde dehydrogenase (asd), which is contained in the pYA292 plasmid. The pYA292 plasmid was also modified, which contains the asd gene, and is therefore capable of rescuing χ4550 S. typhimurium, by incorporating the gene for human IL-2 (pIL2). Thus, there were 2 strains of attenuated S. typhimurium that have been transformed either with the empty pYA292 plasmid (hereafter referred to as attenuated Salmonella lacking the IL-2 gene [Salmonella-no-IL2]), or attenuated Salmonella with the pIL2 plasmid (hereafter referred to as: attenuated Salmonella containing the IL-2 gene, Salmonella-IL2 or SalpIL2). Because the loss of the IL-2-containing pYA292 plasmid (pIL2) would also result in the loss of the plasmid-encoded asd, stable expression of human IL-2 was achieved.

IL-2 produced by Salmonella-IL2 is biologically active. Murine splenocyte NK function was studied as an indicator of in vivo IL-2 biologic activity. Duplicate experiments were conducted in which C57BL/6 mice were randomly divided into 3 groups. Suspensions of Salmonella-IL2 were grown, which exhibited the high IL-2 production to 10¹⁰ CFU/ml; then gavage fed 10⁹ CFU in 0.25 mL to the first group. A suspension of 10⁹ CFU in 0.25 mL was used of the attenuated Salmonella-no-IL2 as a negative control to gavage the second group, and saline to gavage the third group. On days 3 and 7 after gavage, splenic mononuclear cells were isolated using a Ficoll density sedimentation gradient. Interface mononuclear cells were used as effector cells in a 4-hour <51>Cr release cytotoxicity assay against NK-sensitive YAC-1 lymphoma cells. The splenocytes of mice gavage-fed Salmonella-IL2 had significantly increased splenocyte NK activity on days 3 and 7 after gavage, as compared with mice gavage-fed either saline or Salmonella-no-IL2. The findings on day 3 (Table 1) and day 7 (data not shown) did not differ significantly and strongly suggest that the IL-2 produced by Salmonella-IL2 was biologically active.

TABLE 1
Day 3 Effectors
Murine Splenocytes % Cytotoxicity
Control 15.0% ± 6.3
Salmonella-no-IL2 17.3% ± 4.7
Salmonella-IL2 30.5% ± 6.7

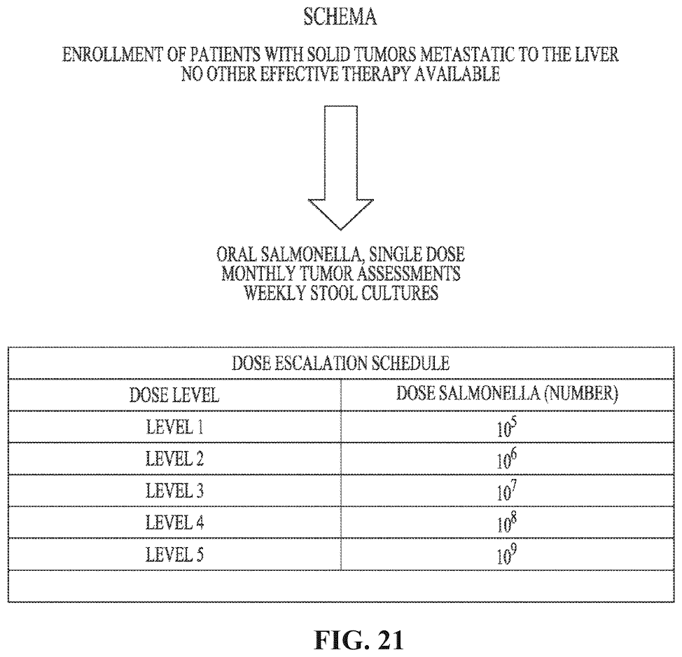

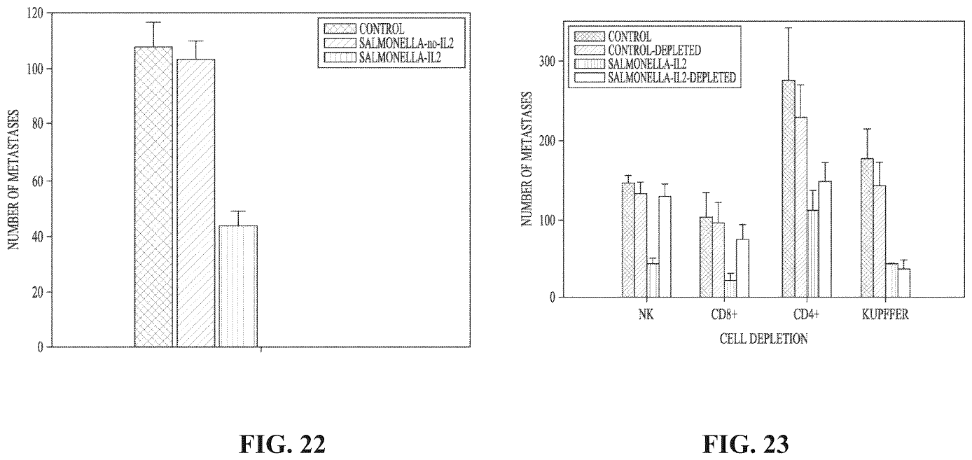

Attenuated Salmonella-IL2 Suppresses In Vivo Tumor Metastases. The in vivo antitumor efficacy of Salmonella-IL2 was evaluated, using murine MCA-38 adenocarcinoma hepatic metastases generated in C57BL/6 mice. The experimental design was based on the Rosenberg model of murine adenocarcinomatous liver metastases; briefly, it consists of performing an intrasplenic injection of 2×10⁵ MCA-38 adenocarcinomatous cells that ultimately result in liver metastases. Mice were divided into 3 groups, 3 days after tumor inoculation and gavage-fed them a single oral dose of (1) saline, (2) Salmonella-no-IL2, or (3) Salmonella-IL2. Surface metastatic deposits were enumerated 14 days after tumor inoculation. As shown in FIG. 21, a significant reduction was found in the number of liver metastases in the Salmonella-IL2 group, as compared with either the control group or the Salmonella-no-IL2 group.

### NK Cells and CD8<+>T Cells are the Major Mediators of the Antitumor Response Elicited by Salmonella-IL2.

The observation that Salmonella-no-IL2 did not demonstrate an antitumor effect led us to surmise that cytokines produced by the host in response to Salmonella colonization alone does not appear to cause these antitumor effects. It was determined which host effector cell population was responsible for the antitumor effect seen with Salmonella-IL2. Since IL-2 directly enhances antitumor cytolytic function of NK cells, macrophages, and T cells and promotes lymphocyte proliferation, any of these cellular mechanisms could contribute to the significant reduction of liver metastases seen with Salmonella-IL2. Standardized treatment model of liver metastases in 4 cohorts of mice was used, (1) untreated control mice gavage-fed saline, (2) untreated control mice gavage-fed saline and depleted of a particular host effector cell population, (3) treated mice gavage-fed Salmonella-IL2, and (4) treated mice gavage-fed Salmonella-IL2 and depleted of a particular host effector cell population. Specific host effector cell populations that were depleted included NK cells, CD8+ cells, CD4+ cells, and liver Kupffer cells. The number of liver metastases was reduced in mice that had been treated with Salmonella-IL2, as compared with control mice treated with saline. This effect was abrogated in mice in which either the NK or the CD8+ effector cells were depleted, but not in mice in which either the CD4+ effector cells or liver Kupffer cells were depleted (FIG. 23). This difference is not surprising because both NK cells and CD8+ cells are known to generate direct tumor destruction. IL-2 markedly potentiates NK cell cytolytic activity against tumor cell targets through the secretion of cytolytic molecules such as perforin and the granzymes. NK cells kill their targets in an MHC unrestricted fashion, while CD8+ cells require the expression of MHC class I by the tumor for effective recognition and killing to take place. CD4+ cells are usually not cytolytic, but have been shown to kill MHC class II tumor targets. Because the tumor used in these studies, MCA-38, does not express MHC class II, it is not surprising that depletion of the CD4+ cell subset did not affect the antitumor response elicited by Salmonella-IL2.

In addition to NK, CD8+, and CD4+ lymphoid cells, the role of the liver Kupffer cells on the antitumor response elicited by Salmonella-IL2 was studied. Salmonella species are facultative intracellular parasites that mainly localize in Kupffer cells and multiply intracellularly. Murine Kupffer cells respond expansively to exogenously administered IL-2 and participate in the induction of antigen-specific immune responses. The depletion of Kupffer cells adversely affected the antitumor response elicited by Salmonella-IL2 was not found.

Attenuated Salmonella invades different tumor cell lines. The unique ability of attenuated Salmonella to maintain immunogenicity after having lost the ability to cause disease has led to more than a decade of study of this organism's therapeutic potential in cancer patients. Salmonella can grow under anaerobic and aerobic conditions, and can survive within epithelial cells as well as macrophages. The precise mechanism of invasion and the antitumor effect seen with attenuated Salmonella in cancer treatment is poorly understood. However, attenuated Salmonella administered intravenously seem to preferentially accumulate within subcutaneously placed tumors, as compared with its normal host reservoir, the liver. Other researchers have replicated these findings and have shown that attenuated Salmonella can halt the growth of subcutaneous tumors. However, how Salmonella accumulate within tumors is not entirely clear. The ability of the attenuated Salmonella-IL2 to invade at 5-, 10-, and 15-minute intervals was investigated, or to multiply within different cancer cell lines: rat hepatocellular carcinoma (Morris7777, hepatoma), mouse neuroblastoma (N2a, neuroblastoma), mouse methylcholanthrene-induced colon carcinoma (MCA-38, colon cancer), and mouse osteosarcoma (K7M2, osteosarcoma). Murine hepatocytes (liver) were used as a positive control and saline as a negative control (control).

Attenuated Salmonella-no-IL2 and Salmonella-IL2 displayed equal invasion efficiencies (percentage of intracellular Salmonella recovered from inocula) into hepatocytes and all cancer cell lines (data not shown). FIG. 23, Panel A shows that the ability of Salmonella-IL2 to invade cultured hepatocytes (liver) was much higher than that of any cancer cell line at all incubation times (p<0.05), displaying a stepwise linear increase in bacterial invasion with increasing bacterial exposure times. Posthoc analysis of the Salmonella-IL2 invasion among cancer cell lines revealed a divergence in invasion efficiency between neuroblastoma and hepatocellular carcinoma as compared with osteosarcoma or human colon cancer cell lines. FIG. 23, Panel B is an expanded portion of FIG. 23, Panel A excluding the data from the murine hepatocyte invasion.

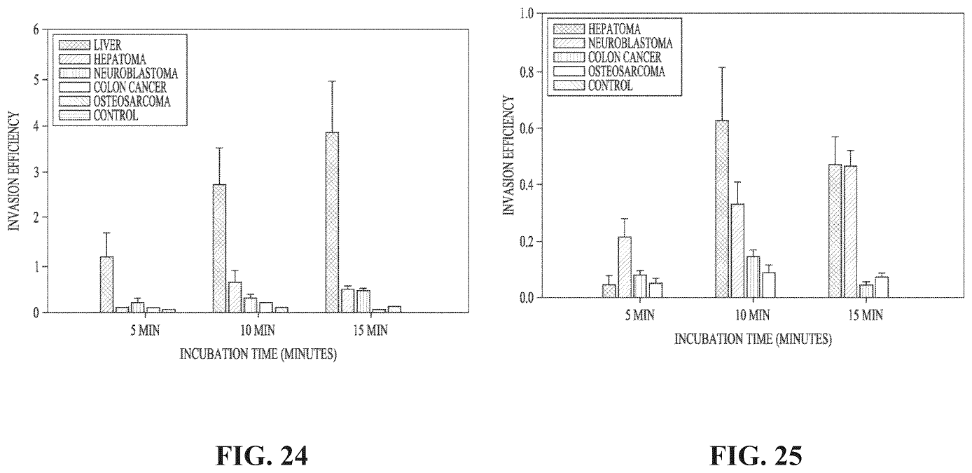

Attenuated Salmonella replicates intracellularly in different tumor cell lines. Once it was observed that attenuated Salmonella differentially invades tumor cells, any differences in intracellular division by this organism were determined within the different tumor cell lines themselves. It was defined that the division efficiency as a measure of intracellular proliferation, infected confluent suspensions of tumor cells with Salmonella-pIL2, and assessed intracellular growth of the bacteria after 24 hours. Again, no difference between the division efficiency of Salmonella-no-IL2 and Salmonella-IL2. FIG. 24 shows that Salmonella-IL2 replicates with greater efficiency within neuroblastoma and osteosarcoma cancer cells, as compared with liver, hepatoma, or colon cancer cells. Overnight incubation revealed greater intracellular division of Salmonella-IL2 in neuroblastoma cells as compared with hepatocytes (liver), hepatoma, or colon cancer cells. The intracellular division of Salmonella-IL2 within osteosarcoma was significantly greater, as compared with hepatoma or colon cancer cells. Therefore, not only do attenuated Salmonella-no-IL2 and Salmonella-IL2 display an enhanced invasion into neuroblastoma and osteosarcoma (as compared with the other malignant cells) but also, the intracellular proliferation of the bacteria is augmented as well.

The invasion of wild-type S. typhimurium into enterocytes is a well-defined process. It was previously demonstrated that attenuated Salmonella-IL2 invasion into the enterocyte cell lines CaCo-2 and HT-29 cells is significantly diminished as compared to its wild-type counterpart. This finding partially accounts for its lower virulence and near absence of Salmonella-associated gastroenteritis. The attenuated Salmonella-IL2 invasion into malignant cell subsets is not as effective as its invasion into hepatocytes, the normal colonization site for this organism. Furthermore, when it was analyzed the invasiveness of Salmonella-IL2, there was a difference between wild-type S. typhimurium and attenuated Salmonella-IL2. Differences in the expression of cell surface proteins may account for this observation. Wild-type S. typhimurium invasion into enterocytes depends on activation of plasma membrane-associated GTP-binding proteins, such as CDC42 and/or Rac1. The binding of Salmonella to these proteins catalyzes a cascade of intracellular signaling that results in membrane ruffling and macropinocytosis of bacteria. Therefore, differences in expression of Rac1 and CDC42 could explain the variability of S. typhimurium invasion into hepatocytes and malignant cell subsets.

Invasive Salmonella-IL2 invasion of neuroblastoma cells is visualized in vivo. It has been shown that Salmonella track to tumor tissue in vitro, and there was also devised a method to directly visualize Salmonella-IL2 invasion of neuroblastoma cells in vivo. Existing attenuated Salmonella strains were modified, Salmonella-no-IL2 and Salmonella-IL2, by transforming these bacteria with a commercially available plasmid containing the green fluorescent protein. Transformants were selected by using the ampicillin resistance conferred by the GFP plasmid. The GFP-transformed bacteria were used in the standard retroperitoneal neuroblastoma model: 14 days after retroperitoneal injection of N2a cells, mice were gavage-fed 10⁸ CFU of either Salmonella-no-IL2-GFP or Salmonella-IL2-GFP. Then, 1 week later, the mice were sacrificed, their tumors enumerated, frozen tissue sections were prepared from the tumors, and the sections were examined under the fluorescent microscope. Only Salmonella-no-IL2-GFP or Salmonella-IL2-GFP constructs grew on agar plates containing ampicillin. Colonies were examined on the ampicillin-containing plates under the fluorescent microscope and found the expected green fluorescence at 488 nm. Intracellular bacteria was found in the tissue sectioned from neuroblastoma extracted from mice treated with Salmonella-no-IL2-GFP or Salmonella-IL2-GFP and excited at 488 nm. Intracellular Salmonella-IL2-GFP bacteria fluoresce more brightly than the surrounding tissue. Small numbers of fluorescent bacteria were found in the liver, spleen, and lungs. Tissue sections examined from mice gavage-fed with non-GFP-transformed Salmonella-no-IL2 and Salmonella-IL2 demonstrated no fluorescence when excited at 488 nm.

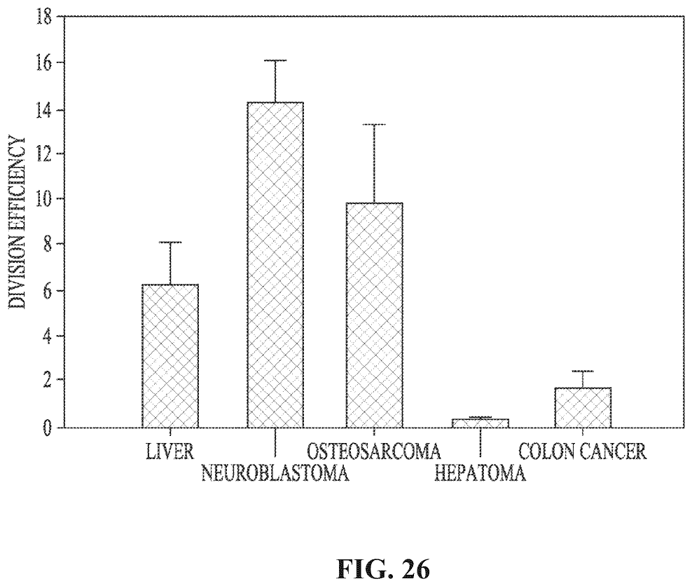

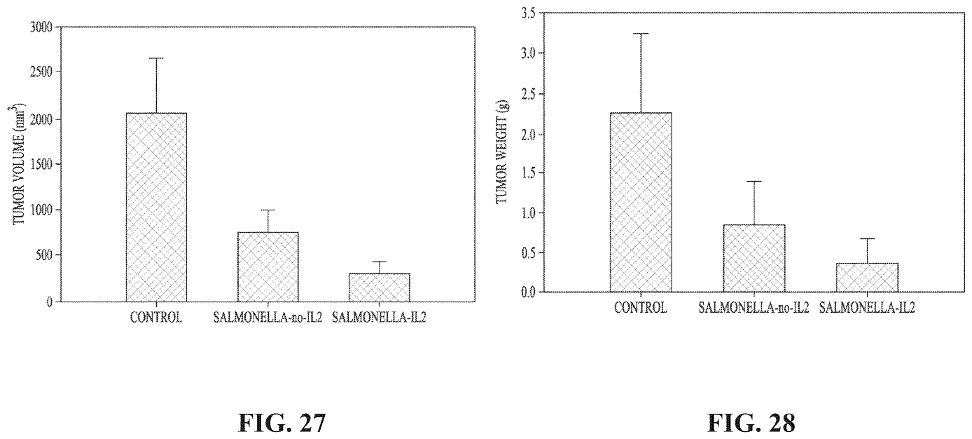

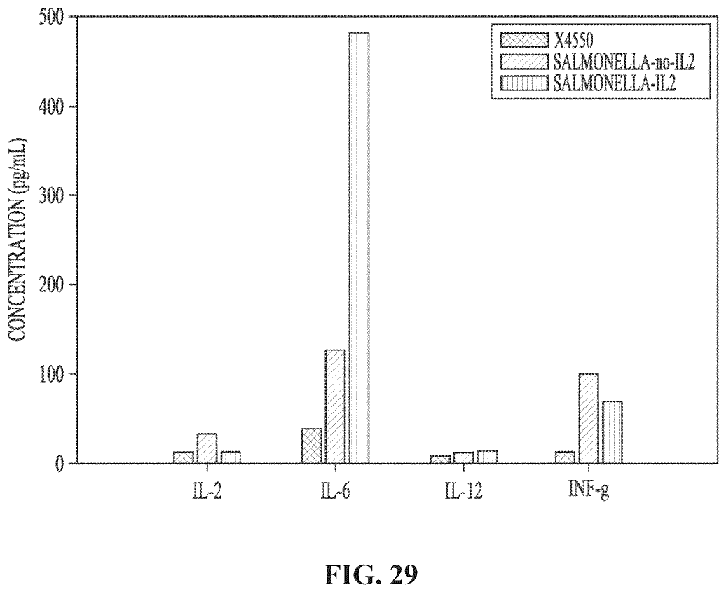

One oral dose of attenuated Salmonella nearly eliminates neuroblastoma. Given the finding of improved attenuated Salmonella invasion and division efficiency in neuroblastoma cells, standard vivo murine model of retroperitoneal neuroblastoma was used and investigated the antitumor effect of attenuated Salmonella. Treatment with Salmonella-no-IL2 and Salmonella-IL2 resulted in a significant reduction in both tumor volume (FIG. 25, Panel A) and tumor weight (FIG. 25, Panel B), as compared with saline controls. Tumor volume is calculated assuming tumor shape as a sphere (4/3 πr<3>). When comparing reductions between mice treated with Salmonella-no-IL2 and Salmonella-IL2, there was a statistically significant decreases in tumor volume and in tumor weight. Cytokines proliferate in response to attenuated Salmonella. In an effort to look more closely at the mechanisms by which attenuated Salmonella affect neuroblastoma tumors, it was evaluated that, in the in vivo retroperitoneal neuroblastoma model, the systemic cytokines, IL-2, IL-6, IL-12, and interferon-gamma (IFN-γ) of cohorts of mice 14 days after treatment with saline (controls), with Salmonella-no-IL2, or with Salmonella-IL2. FIG. 26 shows a dramatic increase in the amount of systemic IL-6 in mice treated with Salmonella-IL2. IL-6 is a pleiotropic cytokine with a variety of immunomodulatory roles, including the activation of cytotoxic T cells and NK cells. It was previously shown that IL-6 is significant in reducing the tumor burden in the model of liver metastases of colon cancer. No upregulation of systemic IL-2 was found, suggesting that the IL-2 produced by Salmonella-IL2 exerts its effect locally.

### Safety of Study Agent

Each of the family of attenuated strains, including the χ4550 strain as described above, given orally to mice at doses up to 10⁹ organisms failed to cause any mortality or morbidity. The genes for cya and crp are widely separated on the salmonella genome making reversion to wild-type for both genes extremely unlikely. When parent strains were grown to late log phase and plated on minimal agar medium with various carbohydrates that should not support growth, occasional mutants were seen with the single deletion (cya-) strain χ4032, and very rare mutants with the double deletion (cya-/crp-) strain χ4064. When these apparent mutants were re-administered to mice they remained avirulent. In humans, a cya-/crp-attenuated salmonella (derived from a different virulent strain) using the asd-balanced lethal vector system (as in χ4550) showed little to no virulence in 22 human volunteers. Over the course of the experiments described above, the study agent has been administered to over 4000 mice without apparent adverse effect. As the study agent has not been administered to humans, this study will provide for first data on human safety.

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount—including any integer—between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

Although the various embodiments have been described with reference to preferred implementations, persons skilled in the art will recognize that changes may be made in form and detail without departing from the spirit and scope thereof.

What is claimed is:

1. A liver treatment agent comprising an effective amount of attenuated Salmonella typhimurium containing a plasmid carrying a coding sequence encoding one or a combination of inhibitors, antagonists or nanobodies capable of reducing fibroid expression.

2. A liver treatment agent comprising an effective amount of attenuated Salmonella typhimurium containing a plasmid carrying a coding sequence encoding a therapeutic composition, said coding sequence directed to one or more of a TGF-β inhibitor, a PDGF inhibitor, a CTGF inhibitor, a TIMP inhibitor, a T-1 receptor antagonist, an NF-κB inhibitor or a periostin inhibitor.

3. The liver treatment agent of claim 2 wherein the coding sequence is directed to a PDGF inhibitor.

4. The liver treatment agent of claim 2 wherein the coding sequence is directed to a CTGF inhibitor.

5. The liver treatment agent of claim 2 wherein the coding sequence is directed to a TIMP inhibitor.

6. The liver treatment agent of claim 2 wherein the coding sequence is directed to an ET-1 receptor antagonist.

7. The liver treatment agent of claim 2 wherein the coding sequence is directed to a NF-κB inhibitor.

8. The liver treatment agent of claim 2 wherein the coding sequence is directed to a periostin inhibitor.

9. The liver treatment agent of claim 2 wherein the coding sequence is directed to a TGF-β inhibitor.

10. The liver treatment agent of claim 2 wherein the agent is capable of reducing fibroid expression.

11. The liver treatment agent of claim 2 wherein the agent is taken as an oral dose.

12. The liver treatment of claim 11 wherein the oral dose is 10⁹ bacteria.

13. A liver treatment agent comprising an effective amount of one or more attenuated Salmonella strains wherein at least one strain contains a plasmid carrying a coding sequence encoding one or a combination of inhibitors, antagonists or nanobodies capable of reducing fibroid expression.

14. The liver treatment agent of claim 13 wherein the Salmonella strains are Salmonella typhimurium strains.

15. The liver treatment agent of claim 14 wherein the agent comprises at least two strains contain a plasmid carrying a coding sequence encoding one or a combination of inhibitors, antagonists or nanobodies capable of reducing fibroid expression, wherein the coding sequence for the at least two strains encode for a different inhibitor, antagonist or nanobody, or a different combination of inhibitors, antagonists or nanobodies.

16. The liver treatment agent of claim 15 wherein the inhibitors, antagonists or nanobodies are selected from the group comprising a TGF-β inhibitor, a PDGF inhibitor, a CTGF inhibitor, a TIMP inhibitor, a T-1 receptor antagonist, an NF-κB inhibitor or a periostin inhibitor.

17. The liver agent of claim 1 wherein the attenuated Salmonella typhimurium containing a plasmid carrying a coding sequence encodes a protein inhibitor.

18. The liver agent of claim 1 wherein the attenuated Salmonella typhimurium containing a plasmid carrying a coding sequence encodes a receptor inhibitor.

19. The liver agent of claim 1 wherein the agent is capable of reducing fibroid expression wherein the fibroid expression is a complication from nonalcoholic steatohepatitis (NASH).

20. The liver agent of claim 1 wherein the agent is capable of reducing fibroid expression wherein the fibroid expression is a complication from alcoholic hepatic cirrhosis.
